(19) United States
(12) Patent Application Publication
(10) Pub. No.: US 2024/0407396 A1
(43) Pub. Date: Dec. 12, 2024

(54) COMBINATION OF BACTERIAL STRAINS TO INHIBIT PATHOGENS IN ANIMALS

(71) Applicant: Phibro Animal Health Corporation, Teaneck, NJ (US)

(72) Inventors: Scott Carter, Teaneck, NJ (US); Kevin Bolek, Teaneck, NJ (US)

(73) Assignee: Phibro Animal Health Corporation, Teaneck, NJ (US)

(21) Appl. No.: 18/732,302

(22) Filed: Jun. 3, 2024

Related U.S. Application Data
(60) Provisional application No. 63/506,527, filed on Jun. 6, 2023.

(51) Int. Cl. A23K 10/18; A23K 50/10; A23K 50/75; A23K 50/80

(52) U.S. Cl. CPC A23K 10/18; A23K 50/10; A23K 50/75; A23K 50/80

(57) ABSTRACT

This disclosure relates to Bacilli combinations consisting essentially of, or consisting of, Bacillus licheniformis, Bacillus pumilus, and Bacillus subtilis. This disclosure also includes compositions of and methods of administering a Bacilli combination further comprising an additional component, such as, for example, a carrier, chemical, feed, feed supplement, plant compound and/or vitamin, and any combinations thereof. In some aspects, compositions can include dry spores, liquid culture, and/or aqueous solutions, and combinations thereof. In some aspects, disclosed compositions treat, prevent, and/or inhibit occurrence of microbial infection in livestock. For example, some aspects include methods and compositions to treat, prevent, and/or inhibit pathogen infections in animals.

### CROSS REFERENCE TO RELATED APPLICATION

This application claims the benefit of the earlier filing date of U.S. Provisional Patent Application No. 63/506,527, filed Jun. 6, 2023, which is incorporated herein by reference in its entirety.

### FIELD

The present application concerns a combination and/or composition comprising three Bacillus species—Bacillus licheniformis, Bacillus pumilus, and Bacillus subtilis—for administration to animals, particularly avians. The present application also concerns the use of these combinations and/or compositions for the prevention, inhibition, and/or treatment of infections caused by pathogens, such as Enterococcus cecorum and/or Clostridium species.

### BACKGROUND

The gastrointestinal (GI) tracts of animals are colonized by a diverse community of microflora. The GI tract may include hundreds of different species and this community profile may change over time based on age and health of the individual. A healthy microbiota community in a subject provides many benefits, such as resistance to pathogens, nutrient absorption, and immune system performance. Intestinal microbiota also play a significant role in mediating pathogenic infections of the gut, which significantly affect quality of life. The gastrointestinal microflora compositions of animals substantially depend largely upon ingested materials. Accordingly, direct-fed microbial (DFM) compositions are commonly administered to influence physiological health.

DFMs can restrict adherence of pathogenic microbes to mucosal surfaces, can stimulate an immune response or proliferation of other endogenous beneficial microorganisms. Moreover, certain DFMs produce and secrete other beneficial compounds or compositions, such as antimicrobial substances. Similar results between feeding DFM products and prophylactic levels of antibiotics for growth have been demonstrated.

### SUMMARY

This disclosure includes a Bacilli combination consisting essentially of Bacillus licheniformis, Bacillus pumilus, and Bacillus subtilis. This Bacilli combination can be beneficial for treating, preventing, or inhibiting pathogens in animals, such as Enterococcus cecorum infections in poultry. Another aspect of this disclosure is a Bacilli combination consisting of Bacillus licheniformis, Bacillus pumilus, and Bacillus subtilis. The Bacillus licheniformis may be, or may comprise, strain OBT 618; the Bacillus pumilus may be, or may comprise, strain OBT 13216; and/or the Bacillus subtilis may be, or may comprise, strain PR 104.

The relative amount of each Bacillus species in the Bacilli combination may be from 1% to 99% of the combination such that the total amount of the Bacillus species in the composition is 100%. In some aspects, the combination comprises from 25% to 75% B. licheniformis, from 25% to 75% B. pumilus, and from 25% to 75% of B. subtilis, such that the total of each species relative to the others is 100%. In other aspects, the combination comprises about 33.3% B. licheniformis, about 33.3% B. pumilus, and about 33.3% B. subtilis. In further aspects, the combination comprises about 20% B. licheniformis, about 30% B. pumilus, and about 50% B. subtilis.

In some aspects, the combination comprises from 10² to 10¹¹ CFU/g of B. licheniformis, from 10² to 10¹¹ CFU/g of B. pumilus, and from 10² to 10¹¹ CFU/g of B. subtilis.

This disclosure also includes a composition comprising a Bacilli combination and an additional component. The additional component in this composition can be a carrier, a vitamin, a copper salt, allicin, alliin, alliinase, algae, a polyphenol or plant material comprising polyphenol, a feed supplement, an additional DFM, a feed, or a combination thereof. In some aspects, the composition does not include an additional Bacillus species. In some aspects, the composition does not include an additional DFM. In other aspects, the additional component can be yucca, quillaja, silica, mineral clay, glucan, inulin, mannans, or endoglucanohydrolase, or any combination thereof.

Additionally disclosed herein is a composition comprising of a Bacilli combination and water. In some aspects, the composition comprising of a Bacilli combination and water can further comprise an acid. In some aspects, the acid comprises acetic acid.

Another aspect is a feed composition for administration to poultry, comprising a Bacilli combination and a poultry feed. The feed composition may further comprise an additional component. In some aspects, the poultry feed comprises a plant material, a carbonate, sulfate, lactate, oxide, propionate, stearate, phosphate, mineral, copper species, sugar, salt, animal protein product, forage product, grain product, plant protein product, processed grain product, roughage product, molasses product, or combinations thereof. In further aspects, the poultry feed can comprise beet pulp, ground corn, corn syrup solids, plant fiber, rice hulls, soluble plant fiber, wheat middlings, microcrystalline cellulose, calcium carbonate, potassium carbonate, potassium sulfate, sodium sulfate, calcium lactate, calcium oxide, calcium propionate, calcium stearate, dicalcium phosphate dehydrate, monocalcium phosphate, sodium tripolyphosphate, tetra sodium pyrophosphate, dolomite, silicon dioxide, silica, limestone, vermiculite, bentonite, montmorillonite, kaolin, glucose, sucrose, dextrose, fructose, maltodextrin, sodium chloride, carrageenan, cellulose, guar gum, polyols, sodium alumino silicate, urea, biotin, folic acid, sodium sesquicarbonate, methionine source, lysine source, L-threonine, or combinations thereof. The poultry feed can additionally comprise a copper sulfate.

Additionally disclosed herein is a method for administering the disclosed Bacilli combination to a subject. The subject can be, for example, livestock and/or aquatic species. In some aspects, the livestock is poultry and/or ruminants. In some examples, the aquatic species is tilapia. This disclosure also includes a method of reducing bird mortality, lesion scores, Enterococcus cecorum, Salmonella species, Escherichia coli, and/or Clostridium perfingens incidence, and/or oocysts in fecal matter comprising administering to poultry an effective amount of any one of the combinations disclosed herein.

The foregoing and other objects, features, and advantages of the aspects will become more apparent from the following detailed description, which proceeds with reference to the accompanying figures.

### BRIEF DESCRIPTION OF THE DRAWINGS

FIG. 1 is a graph of inhibition zone versus Bacillus species, illustrating the average inhibition zone of Bacillus species against E. cecorum strains; error bars show standard deviation. Raw data are presented in Table 1.

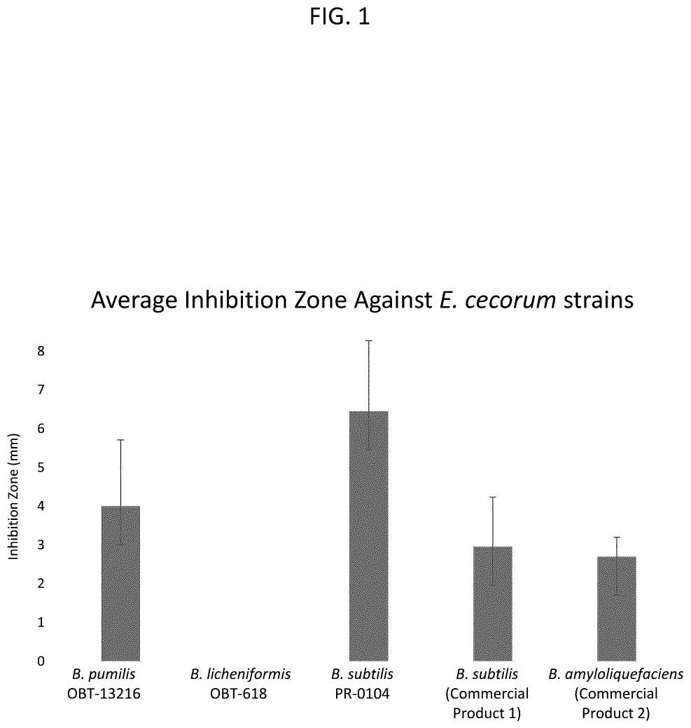

FIG. 2 is a graph of inhibition zone versus Bacillus species, illustrating the average inhibition zone of Bacillus species and strains against E. cecorum strains; error bars show standard deviation. Raw data are presented in Tables 2-5.

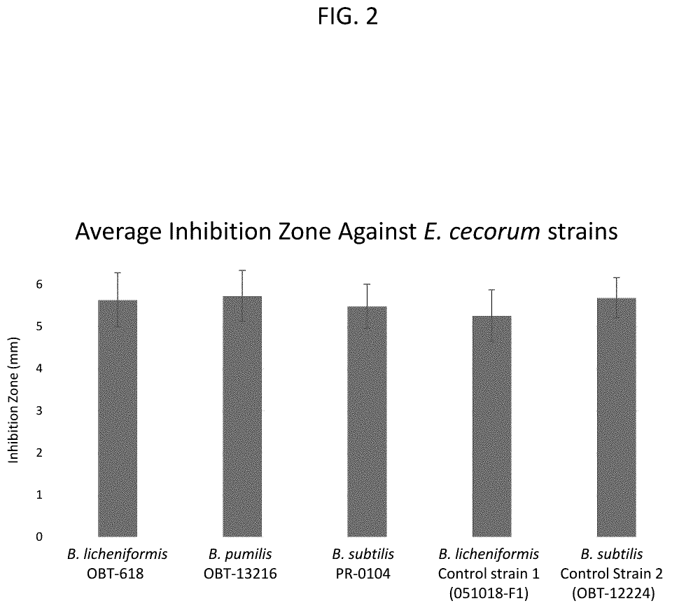

### DETAILED DESCRIPTION

### I. Terms

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. Unless context indicated otherwise, “about” refers to plus or minus 5% of a reference value. For example, “about” 100 refers to 95 to 105. When directly and explicitly distinguishing aspects from discussed prior art, the aspect numbers are not approximates unless the word “about” is recited.

Administering: Administration by any route to a subject, such as poultry, ruminants, or aquatic species.

Antimicrobial: An agent that kills and/or inhibits the growth of microorganisms. As used herein, antimicrobials include antibiotics, antifungals, antivirals, and antiparasitics including anticoccidials, or combinations thereof.

Carrier: A substance that is used as an additive in (or with) a combination, composition, or component as disclosed herein. As used herein, a carrier may be incorporated within particles of a combination, composition, or component, or it may be physically mixed with particles of a combination, composition, or component. A carrier can be used, for example, to modify non-biological properties of a combination or composition, such as flowability, stability during storage, exposure to moisture, etc. Examples of carriers are included herein.

Colony forming units (CFU): “Colony forming units” refers to individual colonies of bacteria. A colony is a mass of individual bacteria growing together. For certain aspects, a colony comprises substantially the same species, and may comprise, but does not necessarily comprise, substantially the same strain. CFU are a measure of the number of bacteria present in or on a surface of a sample. However, CFU are not necessarily a measure of individual cells or spores, as a colony may be formed from a single or a mass of cells or spores.

Combination: A combination includes two or more components that are administered such that the effective time period of at least one component overlaps with the effective time period of at least one other component. A combination, or a component thereof, may be a composition. In some aspects, effective time periods of all components administered overlap with each other. In an exemplary aspect of a combination comprising three components, the effective time period of the first component administered may overlap with the effective time periods of the second and third components, but the effective time periods of the second and third components independently may or may not overlap with one another. In another exemplary aspect of a combination comprising three components, the effective time period of the first component administered overlaps with the effective time period of the second component, but not that of the third component; and the effective time period of the second component overlaps with those of the first and third components. A combination may be a composition comprising the components, a composition comprising one or more components and another separate component (or components) or composition(s) comprising the remaining component(s), or the combination may be two or more individual components. In some aspects, the two or more components may comprise the same component administered at two or more different times, two or more different components administered substantially simultaneously or sequentially in any order, or a combination thereof.

Bacilli Combination: Refers to a combination, or a composition, of DFMs including three Bacillus species selected from Bacillus licheniformis, Bacillus pumilus, and Bacillus subtilis. A Bacilli combination consisting essentially of Bacillus lichemformis, Bacillus pumilus, and Bacillus subtilis does not contain any other Bacillus species but in some aspects, can comprise, for example, an additive, carrier, compound, chemical, food, food supplement, or vitamin. In some disclosed aspects, “Bacilli combination” refers to a composition for administration to a subject, particularly to an animal, and for example, to an avian, such as chickens and turkeys, that consists of or consists essentially of Bacillus licheniformis, Bacillus pumilus, and Bacillus subtilis. In other aspects, “Bacilli combination” refers to Bacillus licheniformis, Bacillus pumilus, and Bacillus subtilis administered in combination without any other DFMs. A person of ordinary skill in the art will understand that the Bacilli combination may include additional residual material that is carried over from the production of any or all of the three Bacillus species, such as a dry milk product, and/or a carrier that does not materially affect the structure, function, novel and/or basic features of the Bacillus species.

Direct fed microbial: A composition that contains live and/or viable microorganisms, typically bacteria and/or yeast, that provides a beneficial effect on an animal.

Feed conversion rate: A measure of the efficiency of an animal to convert feed mass into increased body mass. Typically, the feed conversion rate is calculated as pounds of feed divided by pounds of weight gain, and therefore may be expressed as a dimensionless number. The feed conversion rate is also known in the art as the feed conversion ratio, or feed efficiency.

Mannans: A class of polysaccharides including the sugar mannose. The mannans family includes pure mannans (i.e., the polymer backbone comprises of mannose monomers), glucomannan (the polymer backbone comprises mannose and glucose), and galactomannan (mannans or glucomannan in which single galactose residues are linked to the polymer backbone). Mannans are found in cell walls of some plant species and yeasts, and may be provided as extracts of such plant species and/or yeasts.

Mineral clay: The term “mineral clay” refers to hydrous aluminum silicates. Mineral clays usually include minor amounts of impurities, such as potassium, sodium, calcium, magnesium, and/or iron.

Saponin: A class of chemical compounds, one of many secondary metabolites found in natural sources. Saponins are found in particular abundance in various plant species, such as quillaja and yucca. More specifically, saponins are amphipathic glycosides grouped, in terms of structure, by their composition. In certain aspects, a saponin comprises one or more hydrophilic glycoside moieties combined with a lipophilic triterpene or a triterpene derivative, a steroid or a steroidal derivative, or both.

Strain: A strain refers to two members of the same species having a discernible phenotypic and/or genetic difference.

Subject: Any animal, but particularly livestock (e.g., cows, sheep, goats, pigs, turkeys, and chickens), and aquatic species (e.g., species used in aquaculture such as tilapia). Most typically “subject” refers herein to avians, including poultry, such as chickens and turkeys.

Effective amount: A quantity or concentration of a specified compound, composition or combination sufficient to achieve an effect in a subject.

Vitamin: Includes Vitamin A, Vitamin B₁ (thiamine), Vitamin B₂ (riboflavin), Vitamin B₃ (niacin or niacinamide), Vitamin B₅ (pantothenic acid), Vitamin B₆ (pyridoxine, pyridoxal, or pyridoxamine, or pyridoxine hydrochloride), Vitamin B₇ (biotin), Vitamin B₉ (folic acid), Vitamin B₁₂ (various cobalamins; commonly cyanocobalamin in vitamin supplements), vitamin C, vitamin D, vitamin E, vitamin K, K₁ and K₂ (i.e., MK-4, MK-7), folic acid and biotin, and derivative and analogs thereof.

Additional disclosure is provided by U.S. patent application Ser. No. 14/699,740, U.S. patent application Ser. No. 13/566,433, U.S. patent application Ser. No. 13/872,935, U.S. Patent Publication No. 2013/0017211, U.S. Patent Publication No. 2012/0156248, U.S. Patent Publication No. 2007/0253983, U.S. Patent Publication No. 2007/0202092, U.S. Patent Publication No. 2007/0238120, U.S. Patent Publication No. 2006/0239992, U.S. Patent Publication No. 2005/0220846, U.S. Patent Publication No. 2005/0180964, and Australian Patent Application No. 2011/201420, each of which is incorporated herein by reference in its entirety.

### II. Overview

Enterococcus cecorum is an emerging pathogen in poultry globally. The bacterial species is traditionally considered a commensal bacterium in the gastrointestinal tract of animals, but rates of pathogenic E. cecorum strains in poultry are rising. E. cecorum is a gram-positive facultative anaerobe and is increasingly recognized a causative agent of enterococcal spondylitis in chickens. A primary symptom of enterococcal spondylitis is locomotor dysfunction resulting from femoral head necrosis. Outbreaks result in high morbidity and mortality, leading to high culling, carcass condemnations, and significant economic loss for poultry industries. Due to E. cecorum's recent emergence as a devastating pathogen, best control practices have yet to be established.

Clostridia species are other problematic bacterial pathogens in agricultural industries, including in ruminants production. These bacteria are gram-positive anaerobes and generally considered opportunistic pathogens. Infections can develop in an animal after the animal consumes contaminated feed or via an open wound. Symptoms of C. perfringens infection in poultry include diarrhea, intestinal lesions, decreased nutritional absorption, and weight loss, and can cause mortality rates of up to 50%. Symptoms of Clostridium perfringens infection in cattle include gut distress, hemorrhagic bowel syndrome, stalled calves, blindness, and sudden death. Effective control practices to prevent Clostridia infections are important to agricultural livestock industries.

### III. Bacilli Combination

A Bacilli combination is a combination or composition comprising, consisting essentially of, or consisting of, three Bacillus species, selected from Bacillus licheniformis, Bacillus pumilus, and Bacillus subtilis. In some aspects, the Bacilli combination consists essentially of Bacillus licheniformis, Bacillus pumilus, and Bacillus subtilis. That is, the Bacilli combination does not include any additional Bacillus species other than the Bacillus licheniformis, Bacillus pumilus, and Bacillus subtilis. In other aspects, the Bacilli combination consists of Bacillus licheniformis, Bacillus pumilus, and Bacillus subtilis. In some aspects, the Bacilli combination does not include any additional DFM.

Certain aspects of the present disclosure concern the discovery that administering the disclosed Bacilli combination to a subject provides a substantial benefit to the subject compared to a subject that is not administered the combination. With particular reference to poultry, the disclosed Bacilli combination provides a substantial benefit to the poultry, such as with respect to Enterococcus cecorum, Salmonella spp., Escherichia coli, and/or Clostridium perfingens (CP) incidence, and/or one or more of feed conversion rate, average body weight, average body weight gain, body weight coefficient of variation, bird mortality, lesion scores, and/or oocysts in fecal matter relative to poultry fed none, one, or two of these Bacilli in any combination.

### A. Bacillus Strains

A person of ordinary skill in the art will appreciate that any strain, or combination of strains, of Bacillus licheniformis, Bacillus pumilus, and Bacillus subtilis can be used in the Bacilli combination. As used herein the terms “Bacillus licheniformis,” “Bacillus pumilus,” and “Bacillus subtilis” independently may refer to a single strain of the respective Bacillus species, or to multiple strains, such as 2, 3, 4, 5, 6, 7, 8, 9, 10 or more strains, of each respective Bacillus species. In certain aspects, the Bacilli combination includes a single strain of Bacillus licheniformis, a single strain of Bacillus pumilus, and a single strain of Bacillus subtilis. Solely by way of example and without limitation, certain acceptable exemplary strains of each Bacillus species are listed below.

### Bacillus licheniformis Strains

Bacillus licheniformis (Weigmann) Chester ATCC® 6598™ strain NRS 745 deposited with ATCC as Bacillus subtilis by (Ehrenberg) Cohn, Bacillus licheniformis (Weigmann) Chester ATCC® 6634™ strain NRS 304, Bacillus licheniformis (Weigmann) Chester ATCC® 8480™ strain NRS 1128, Bacillus licheniformis (Weigmann) Chester ATCC® 9259™, Bacillus licheniformis (Weigmann) Chester ATCC® 9789™ strain AMNH 723, ATCC 102, ATCC 4527, ATCC 8243, ATCC 9800, NCTC 2586, NCTC 6346, NRS 243, NRS 978, W. Ford 1, DSM 8785, DSM 46308, BU 171, CCDB b-30, CCEB 631, CCM 2205, CN 1060, HNCMB 101012, IFO 12195, IFO 12196, IMET 11025, NBRC 12195, NBRC 12196, NCDO 735, NCDO 835, NCIB 6346, NCIB 8059, NCIB 8061, OUT 8367, OUT 8368, Smith 243, Smith 978, HankeyB13 deposited with ATCC as Bacillus subtilis by (Ehrenberg) Cohn, Bacillus licheniformis (Weigmann) Chester ATCC® 9945™ strain NRS 712, NCIB 8062 deposited with ATCC as Bacillus subtilis by (Ehrenberg) Cohn, Bacillus licheniformis (Weigmann) Chester ATCC® 9945a™ strain CD-2, NCIB 11709, Bacillus licheniformis (Weigmann) Chester ATCC® 10716™ strain ATCC 11944, BS 2181, Boots 1343, CCM 2181, FDA BT1, NCIB 8874, NRS 1330, Tracy I, DSM 603, IFO 12199, NBRC 12199, Bacillus licheniformis (Weigmann) Chester ATCC® 11945™ strain 1331, FDA BT3, Bacillus licheniformis (Weigmann) Chester ATCC® 11946™ strain 1333, B-1001, Bacillus licheniformis (Weigmann) Chester ATCC® 12139™ strain CSC deposited with ATCC as Bacillus subtilis by (Ehrenberg) Cohn, Bacillus licheniformis (Weigmann) Chester ATCC® 12713™ strain PRL B479, NRRL B-1001, Bacillus licheniformis (Weigmann) Chester ATCC® 12759™ strain ATCC 11560, Damodaron P-8, LMG 7560, NRS 1415, Vitek #200148, NCIB 8549, HankeyB133, P8, Bacillus licheniformis (Weigmann) Chester ATCC® 12759-MINI-PACK™ strain ATCC 11560, Damodaron P-8, LMG 7560, NRS 1415, Vitek #200148, Bacillus licheniformis (Weigmann) Chester ATCC® 13438™ Strain NCTC 8233, M. II strain, Bacillus licheniformis (Weigmann) Chester ATCC® 14409™ strain 620, NRS 1114, NCIB 1042, deposited with ATCC as Bacillus abysseus by ZoBell and Upham, Bacillus licheniformis (Weigmann) Chester ATCC® 14580™ strain (Gibson) 46, NCIB 9375, NCTC 10341, NRS 1264, DSM 13, CCM 2145, IFO 12200, NBRC 12200, WDCM 00068, Bacillus licheniformis (Weigmann) Chester ATCC® 14580D-5™ strain designation: Genomic DNA from Bacillus licheniformis Strain 46 [ATCC® 14580™]Bacillus licheniformis (Weigmann) Chester ATCC® 14594™, Bacillus licheniformis (Weigmann) Chester ATCC® 21038™ strain L-065, Bacillus licheniformis (Weigmann) Chester ATCC® 21039™, Bacillus licheniformis (Weigmann) Chester ATCC® 21415™ strain NS 1 deposited with ATCC as Bacillus subtilis by (Ehrenberg) Cohn, Bacillus licheniformis (Weigmann) Chester ATCC® 21417™ strain M deposited with ATCC as Bacillus subtilis (Ehrenberg) Cohn, Bacillus licheniformis (Weigmann) Chester ATCC® 21418™ deposited with ATCC as Bacillus subtilis by (Ehrenberg) Cohn, Bacillus licheniformis (Weigmann) Chester ATCC® 21424™ strain DSM 1969, Bacillus licheniformis (Weigmann) Chester ATCC® 21610™ strain B-201-7 deposited with ATCC as Bacillus subtilis (Ehrenberg) Cohn, Bacillus licheniformis (Weigmann) Chester ATCC® 21667™ strain FD 23612, Bacillus licheniformis (Weigmann) Chester ATCC® 21733™ strain DSM 1913 deposited with ATCC as Bacillus subtilis by (Ehrenberg) Cohn, Bacillus licheniformis (Weigmann) Chester ATCC® 25972™ strain 749/C, DSM 8782, DSM 46217, IMET10723, NCIB 9443, Bacillus licheniformis (Weigmann) Chester ATCC® 27326™ strain OM-81, Bacillus licheniformis (Weigmann) Chester ATCC® 27811™ strain 584, FERM-P 1038, Bacillus licheniformis (Weigmann) Chester ATCC® 31667™ strain DG 14, Bacillus licheniformis (Weigmann) Chester ATCC® 31972™ strain PM-3, Bacillus licheniformis (Weigmann) Chester ATCC® 33632™ strain (JOC) 2390, NCIB 11672, Bacillus licheniformis (Weigmann) Chester ATCC® 39326™, Bacillus licheniformis (Weigmann) Chester ATCC® 53757™ strain PWD-1, Bacillus licheniformis (Weigmann) Chester ATCC® 53926™ strain E312, Bacillus licheniformis (Weigmann) Chester ATCC® 55768™ strain O.W.U. 138B [OWU 138B], Bacillus licheniformis (Weigmann) Chester strain DSM 15, C, Bacillus licheniformis (Weigmann) Chester strain DSM 392, Bacillus licheniformis (Weigmann) Chester strain DSM 394, Bacillus licheniformis (Weigmann) Chester strain DSM 7259, NRRL-NRS 1263, Bacillus licheniformis (Weigmann) Chester strain DSM 7459, Bacillus licheniformis (Weigmann) Chester strain DSM 11258, Bacillus licheniformis (Weigmann) Chester strain DSM 11259, Bacillus licheniformis (Weigmann) Chester strain DSM 12369, Bacillus licheniformis (Weigmann) Chester strain DSM 12370, Bacillus licheniformis (Weigmann) Chester strain DSM 26543, Bacillus licheniformis (Weigmann) Chester strain DSM 28096, Bacillus licheniformis (Weigmann) Chester strain DSM 28591, Bacillus licheniformis (Weigmann) Chester strain DSM 30523, Bacillus licheniformis (Weigmann) Chester strain DSM 30535, Bacillus licheniformis (Weigmann) Chester strain DSM 30542, Bacillus licheniformis (Weigmann) Chester strain DSM 30585, Bacillus licheniformis (Weigmann) Chester strain DSM 30615, Bacillus licheniformis (Weigmann) Chester strain DSM 30620, Bacillus licheniformis (Weigmann) Chester strain DSM 30624, Bacillus licheniformis (Weigmann) Chester strain DSM 30643, Bacillus licheniformis (Weigmann) Chester strain DSM 30654, Bacillus licheniformis (Weigmann) Chester strain DSM 30724, Bacillus licheniformis (Weigmann) Chester strain DSM 30766, Bacillus licheniformis (Weigmann) Chester strain DSM 30769, Bacillus licheniformis (Weigmann) Chester strain DSM 30778, Bacillus licheniformis (Weigmann) Chester strain DSM 30779, Bacillus licheniformis (Weigmann) Chester strain DSM 30865, Bacillus licheniformis (Weigmann) Chester strain DSM 30926, Bacillus licheniformis (Weigmann) Chester strain DSM 30959, Bacillus licheniformis (Weigmann) Chester strain DSM 30960, Bacillus licheniformis (Weigmann) Chester strain DSM 30961, Bacillus licheniformis (Weigmann) Chester strain DSM 30976, Bacillus licheniformis (Weigmann) Chester strain DSM 31019, Bacillus licheniformis (Weigmann) Chester strain DSM 100653, Bacillus licheniformis (Weigmann) Chester strain DSM 100655, Bacillus licheniformis (Weigmann) Chester strain DSM 103059, Bacillus licheniformis (Weigmann) Chester strain NCIB 1525, 1229, Bacillus licheniformis (Weigmann) Chester strain NCIB 6816, Glaxo 417, Bacillus licheniformis (Weigmann) Chester strain NCIB 7224, Loos, Bacillus licheniformis (Weigmann) Chester strain NCIB 8536, P1, Bacillus licheniformis (Weigmann) Chester strain NCIB 8537, Ho, Bacillus licheniformis (Weigmann) Chester strain NCIB 9536, Gibson 1319, NRS 1553, Bacillus licheniformis (Weigmann) Chester strain NCIB 9667, 1, Bacillus licheniformis (Weigmann) Chester strain NCIB 9668, 2, Bacillus licheniformis (Weigmann) Chester strain NCIB 9669, 3, Bacillus licheniformis (Weigmann) Chester strain NCIB 10689, Bacillus licheniformis (Weigmann) Chester strain NCIB 11143, Bacillus licheniformis (Weigmann) Chester strain NCIB 11643, YNS7712R, Bacillus licheniformis (Weigmann) Chester strain NCIB 13497, Bacillus licheniformis (Weigmann) Chester strain NCIB 14014, DA33, Bacillus licheniformis B1 (NRRL Deposit Number B-50907), Bacillus subtilis B2 (Deposit Number B-50908), Bacillus licheniformis RW25 (NRRL Deposit Number B-50911), Bacillus licheniformis RW32 (NRRL Deposit Number B-50912), and Bacillus licheniformis RW41 (NRRL Deposit Number B-50913), Bacillus licheniformis BL21 (NRRL B-50134), Bacillus licheniformis 3-12a (NRRL B-50504), Bacillus licheniformis 4-2a (NRRL B-50506), Bacillus licheniformis 842 (NRRL B-50516), Bacillus licheniformis DSM 5749 (BioPlus® 2B, Chr. Hansen Bio Systems), and Bacillus licheniformis OBT618 (NRRL-B-233118).

### Bacillus pumilus Strains

Bacillus pumilus OBT 13216 (ATCC number or NRRL-B-68101).

### Bacillus subtilis Strains

Bacillus subtilis (Ehrenberg) Cohn ATCC®82™ strain AMC, ATCC 8037, NRS 315, Bacillus subtilis (Ehrenberg) Cohn ATCC®82D-5™ strain designation: Genomic DNA from Bacillus subtilis strain AMC [ATCC® 82™], Bacillus subtilis (Ehrenberg) Cohn ATCC®465™ strain NRS 743, Bacillus subtilis (Ehrenberg) Cohn ATCC®4529™ strain 3, ATCC 8013, NCTC 2588, NRS 1004 deposited with ATCC as Bacillus vulgatus by Trevisan, Bacillus subtilis (Ehrenberg) Cohn ATCC®4925™ strain NRS 740 deposited with ATCC as Bacillus nigrificans by Fabian and Nienhuis, Bacillus subtilis (Ehrenberg) Cohn ATCC®4944™ strain NCTC, NRS 1106 deposited with ATCC as Bacillus parvus, Bacillus subtilis subspecies subtilis (Ehrenberg) Cohn ATCC® 6051™ strain Marburg strain, ATCC 6051-U, CCM 2216, CCRC 10255, CCUG 163B, CFBP 4228, CIP 52.65, DSM 10, IAM 12118, IFO 12210, IFO 13719, IFO 16412, IMET 10758, JCM 1465, LMG 7135, NCAIM B.01095, NCCB 32009, NCCB 53016, NCCB 70064, NCFB 1769, NCIB 3610, NCTC 3610, NRRL B-4219, NRS 1315, NRS 744, VKM B-501, NBRC 13719 deposited with ATCC as Bacillus subtilis (Ehrenberg) Cohn, Bacillus subtilis (Ehrenberg) Cohn ATCC®6051a™ strain P31K6, Bacillus subtilis bacteriophage phi-e ATCC®6051-B1™ strain Phi-e deposited with ATCC as phi e, Bacillus subtilis (Ehrenberg) Cohn ATCC®6460™ strain NRS 259 deposited with ATCC as Bacillus aterrimus by Lehmann and Neumann, Bacillus subtilis (Ehrenberg) Cohn ATCC®6461™ strain NRS 275, CN 2192, NCIB 8055 deposited with ATCC as Bacillus aterrimus by Lehmann and Neumann, Bacillus subtilis subspecies spizizenii Nakamura et al. ATCC® 6633™ strain NRS 231, DSM 347, CCM 1999, IAM 1069, NCIB 8054, NCTC 10400, WDCM 00003 deposited with ATCC as Bacillus subtilis (Ehrenberg) Cohn, Bacillus subtilis subspecies spizizenii Nakamura et al. ATCC® 6633D-5™ strain designation: Genomic DNA from Bacillus subtilis subspecies spizizenii strain NRS 231 [ATCC®6633™] deposited with ATCC as Bacillus subtilis (Ehrenberg) Cohn, Bacillus subtilis subspecies spizizenii Nakamura et al. ATCC® CRM-6633™ strain NRS 231 deposited with ATCC as Bacillus subtilis (Ehrenberg) Cohn, Bacillus subtilis subspecies spizizenii Nakamura et al. ATCC® 6633-MINI-PACK™ strain NRS 231 deposited with ATCC as Bacillus subtilis (Ehrenberg) Cohn, Bacillus subtilis (Ehrenberg) Cohn ATCC®6984™ strain NRS 747 deposited with ATCC as Bacillus vulgatus subspecies hydrolyticus, Bacillus subtilis (Ehrenberg) Cohn ATCC®7003™ strain NRS 730, Bacillus subtilis (Ehrenberg) Cohn ATCC®7058™ strain NRS 351, Bacillus subtilis (Ehrenberg) Cohn ATCC®7059™ strain NRS 352, Bacillus subtilis (Ehrenberg) Cohn ATCC®7060™ strain NRS 659, Bacillus subtilis (Ehrenberg) Cohn ATCC®7067™ strain NRS 238, ATCC 7974, ATCC 8012, Bacillus subtilis (Ehrenberg) Cohn ATCC®7480™ strain NRS 1107 deposited with ATCC as Bacillus endoparasiticus by (Benedek) Benedek, Bacillus subtilis (Ehrenberg) Cohn ATCC®8188™ strain ATCC 8450, NRS 773 deposited with ATCC as Tyrothrix minimus, Bacillus subtilis (Ehrenberg) Cohn ATCC®8473™ strain NRS 762, Bacillus subtilis (Ehrenberg) Cohn ATCC®9466™ strain designation: FDA strain PCI 220 [BUCSAV 170, NCIB 8159, NRRL B-558, NRS 1088], Bacillus subtilis (Ehrenberg) Cohn ATCC®9524™ strain 3R9675, NRS 1109, Bacillus subtilis (Ehrenberg) Cohn ATCC®9799™ strain NCTC 6276, NRS 1125, Bacillus subtilis (Ehrenberg) Cohn ATCC®9858™ strain NRS 237, NCIB 8063, Bacillus subtilis (Ehrenberg) Cohn ATCC®9943™ strain NRS 979, Bacillus subtilis (Ehrenberg) Cohn ATCC®10774™ strain BU169, NCIB 8872, Bacillus subtilis (Ehrenberg) Cohn ATCC®10783™ strain NRRL B-543, Bacillus subtilis (Ehrenberg) Cohn ATCC®11774™ strain NCTC 8236, DSM 2109, Bacillus subtilis (Ehrenberg) Cohn ATCC® 11838™ strain AMC 46-A-6 (strain I), NCIB 8850, Bacillus subtilis (Ehrenberg) Cohn ATCC®12100™ strain NCA 1558, ND 957, Bacillus subtilis (Ehrenberg) Cohn ATCC®12432™ strain MB 32, 56R188, ATCC 13597, NCIB 8993, Bacillus subtilis (Ehrenberg) Cohn ATCC®12695™ strain 51-52, Bacillus subtilis (Ehrenberg) Cohn ATCC®12711™ strain PRL B92, Ra, Bacillus subtilis (Ehrenberg) Cohn ATCC®13542™, Bacillus subtilis (Ehrenberg) Cohn ATCC®13933™ strain NRRL B-1471, Bacillus subtilis (Ehrenberg) Cohn ATCC®13952™ strain 1346, Bacillus subtilis (Ehrenberg) Cohn ATCC®14410™ strain 625, NRS 1115 deposited with ATCC as Bacillus borborokoites by ZoBell and Upham, Bacillus subtilis (Ehrenberg) Cohn ATCC®14415™ strain 569, NRS 1120 deposited with ATCC as Bacillus submarinus by ZoBell and Upham, Bacillus subtilis (Ehrenberg) Cohn ATCC®14416™ strain 576, NRS 1121 deposited with ATCC as Bacillus thalassokoites by ZoBell and Upham, Bacillus subtilis (Ehrenberg) Cohn ATCC®14593™ strain IAM 1145, Bacillus subtilis (Ehrenberg) Cohn ATCC®14617™ strain A-1625, Bacillus subtilis (Ehrenberg) Cohn ATCC®14660™ strain C30-1, Bacillus subtilis (Ehrenberg) Cohn ATCC®14662™ strain C30-109, Bacillus subtilis (Ehrenberg) Cohn ATCC®14807™ strain MB-155, Bacillus subtilis (Ehrenberg) Cohn ATCC®15040™ strain SX-67, Bacillus subtilis (Ehrenberg) Cohn ATCC®15041™ strain SX-92, Bacillus subtilis (Ehrenberg) Cohn ATCC®15134™ deposited with ATCC as Bacillus uniflagellatus by Mann, Bacillus subtilis (Ehrenberg) Cohn ATCC®15183™ strain 309, Bacillus subtilis (Ehrenberg) Cohn ATCC®15244™ strain 3369, Bacillus subtilis (Ehrenberg) Cohn ATCC®15245™ strain 3349, IAM 1-3 deposited with ATCC as Bacillus natto by Sawamura, Bacillus subtilis (Ehrenberg) Cohn ATCC®15476™ strain M-4-45, Bacillus subtilis (Ehrenberg) Cohn ATCC®15477™ strain M-24-1 deposited with ATCC as Bacillus pumilus by Meyer and Gottheil, Bacillus subtilis (Ehrenberg) Cohn ATCC®15561™ strain K-X-1, A-1, Bacillus subtilis (Ehrenberg) Cohn ATCC®15563™ strain Marburg, Bacillus subtilis bacteriophage SP8 ATCC®15563-B1™ strain SP8 deposited with ATCC as SP8 bacteriophage, Bacillus subtilis (Ehrenberg) Cohn ATCC®15575™ strain SB 19, Bacillus subtilis (Ehrenberg) Cohn ATCC®15811™ strain 5380, Bacillus subtilis (Ehrenberg) Cohn ATCC®15818™ strain RIA 445, Bacillus subtilis (Ehrenberg) Cohn ATCC®15819™ strain RIA 447, Bacillus subtilis (Ehrenberg) Cohn ATCC®15841™, Bacillus subtilis bacteriophage S-a ATCC®15841-B1™ strain S-a deposited with ATCC as S-a bacteriophage, Bacillus subtilis (Ehrenberg) Cohn ATCC®19659™ strain PRD 66, IFO 13722, Bacillus subtilis (Ehrenberg) Cohn ATCC®19659-MINI-PACK™ strain PRD 66, IFO 13722, Bacillus subtilis (Ehrenberg) Cohn ATCC®21008™ strain 182-H-86 deposited with ATCC as Bacillus pumilus by Meyer and Gottheil, Bacillus subtilis (Ehrenberg) Cohn ATCC®21183™ strain 5221, Bacillus subtilis (Ehrenberg) Cohn ATCC®21228™ strain SC 8548, SO-4, DSM 1970, Bacillus subtilis (Ehrenberg) Cohn ATCC®21331™ strain IFO 35, Bacillus subtilis (Ehrenberg) Cohn ATCC®21332™ strain IAM 1213, Bacillus subtilis (Ehrenberg) Cohn ATCC®21394™ strain 4-3-Ky, DSM 1971 deposited with ATCC as Bacillus subtilis subspecies sakainensis, Bacillus subtilis (Ehrenberg) Cohn ATCC®21555™ strain Y 13, Bacillus subtilis (Ehrenberg) Cohn ATCC®21556™, Bacillus subtilis (Ehrenberg) Cohn ATCC®21742™ strain AHr-5, Bacillus subtilis (Ehrenberg) Cohn ATCC®21770™ strain SP-3 deposited with ATCC as Bacillus cereus by Frankland and Frankland, Bacillus subtilis (Ehrenberg) Cohn ATCC®21951™ strain 716, IFO 13322 deposited with ATCC as Bacillus pumilus by Meyer and Gottheil, Bacillus subtilis (Ehrenberg) Cohn ATCC®23059™ strain W23, Bacillus subtilis (Ehrenberg) Cohn ATCC®23856™ strain EMG 50, SB19, Bacillus subtilis (Ehrenberg) Cohn ATCC®23857™ strain 168, Bacillus subtilis (Ehrenberg) Cohn ATCC®23857D-5™ strain Designation: Genomic DNA from Bacillus subtilis strain 168 [ATCC® 23857™], Bacillus subtilis (Ehrenberg) Cohn ATCC®23858™ strain EMG 52, Bacillus subtilis (Ehrenberg) Cohn ATCC®23859™ strain EMG 53, Bacillus subtilis (Ehrenberg) Cohn ATCC®25369™ strain 24028 deposited with ATCC as Bacillus pulvifaciens by Nakamura, Bacillus subtilis (Ehrenberg) Cohn ATCC®27328™ strain C, Bacillus subtilis (Ehrenberg) Cohn ATCC®27370™ strain 168 M, Bacillus subtilis bacteriophage SPO1 ATCC®27370-B1™ strain SPO1 deposited with ATCC as SPO1, Bacillus subtilis (Ehrenberg) Cohn ATCC®27505™ strain K49, HER 1346 deposited with ATCC as Bacillus subtilis subspecies amyloliquefaciens, Bacillus subtilis (Ehrenberg) Cohn ATCC®27689™ strain SB168 (trp-), Bacillus subtilis (Ehrenberg) Cohn ATCC®29056™ strain SB100, Bacillus subtilis (Ehrenberg) Cohn ATCC®29233™ strain X6, Bacillus subtilis (Ehrenberg) Cohn ATCC®31002™ strain Ahr.AUr-9, FERM-1998, Bacillus subtilis (Ehrenberg) Cohn ATCC®31028™ strain FD 6404 deposited with ATCC as Bacillus globigii by Migula, Bacillus subtilis (Ehrenberg) Cohn ATCC®31091™ strain 1054, IFO 13586, Bacillus subtilis (Ehrenberg) Cohn ATCC®31094™ strain 1097, IFO 13621, Bacillus subtilis (Ehrenberg) Cohn ATCC®31098™ strain 1027, IFO 13585 deposited with ATCC as Bacillus pumilus by Meyer and Gottheil, Bacillus subtilis subspecies subtilis (Ehrenberg) Nakamura et al. ATCC®31578™ strain DSM 6223, RUB 331, Bacillus subtilis (Ehrenberg) Cohn ATCC®31954™ strain MO7S-16/11, Bacillus subtilis (Ehrenberg) Cohn ATCC®33234™ strain NCIB 10106, Bacillus subtilis (Ehrenberg) Cohn ATCC®35021™ strain 5230, NRS 6, Bacillus subtilis (Ehrenberg) Cohn ATCC®35854™ strain NRRL B-3411, Bacillus subtilis (Ehrenberg) Cohn ATCC®35946™ strain OSU 75, Bacillus subtilis subspecies subtilis (Ehrenberg) Nakamura et al. ATCC®37014™ strain DSM 6224, BD170, pSA2100, Bacillus subtilis subspecies subtilis (Ehrenberg) Nakamura et al. ATCC®37015™ strain DSM 4514, BD170, NCIB 11624, pUB110, Bacillus subtilis subspecies subtilis (Ehrenberg) Nakamura et al. ATCC®37108™ strain DSM 4873, BGSC 1E32, BR151, pPL608, Bacillus subtilis subspecies subtilis (Ehrenberg) Nakamura et al. ATCC®37128™ strain DSM 4554, BGSC 1E18, pE194, Bacillus subtilis subspecies subtilis (Ehrenberg) Nakamura et al. ATCC®39090™ strain DSM 6198, BGSC 1S53, Bacillus subtilis (Ehrenberg) Cohn ATCC®39320™ strain MB 4488, Bacillus subtilis (Ehrenberg) Cohn ATCC®39374™ strain MB 3575, Bacillus subtilis (Ehrenberg) Cohn ATCC®39706™ strain B1-20, Bacillus subtilis (Ehrenberg) Cohn ATCC®43223™ strain ABM261, Bacillus subtilis (Ehrenberg) Cohn ATCC®49343™ strain IMVS 0101, Bacillus subtilis (Ehrenberg) Cohn ATCC®49760™ deposited with ATCC as Bacillus globigii by Migula, Bacillus subtilis (Ehrenberg) Cohn ATCC®49822™ deposited with ATCC as Bacillus globigii by Migula, Bacillus subtilis (Ehrenberg) Cohn ATCC®55033™ strain SMS274, Bacillus subtilis (Ehrenberg) Cohn ATCC®55060™ strain MB 4974, Bacillus subtilis (Ehrenberg) Cohn ATCC®55405™ strain 300, Bacillus subtilis subspecies inaquosorum ATCC®55406™ strain DA33 deposited with ATCC as Bacillus licheniformis (Weigmann) Chester, Bacillus subtilis (Ehrenberg) Cohn ATCC®55422™ strain SC 15257, Bacillus subtilis (Ehrenberg) Cohn ATCC®55614™ strain 1.2, AQ153, Bacillus subtilis (Ehrenberg) Cohn ATCC®55675™ strain BP01, Bacillus subtilis subspecies subtilis (Ehrenberg) Nakamura et al. strain DSM 402, BRC 111470, NCIB 10106, Bacillus subtilis subspecies spizizenii Nakamura et al. strain DSM 618, Bacillus subtilis subspecies spizizenii Nakamura et al. strain DSM 1087, Bacillus subtilis (Ehrenberg) Cohn strain DSM 1088, IFO 13169, NBRC 13169, OUT 8353, Bacillus subtilis (Ehrenberg) Cohn strain DSM 1089, IFO 3026, NBRC 3026, OUT 8350, Bacillus subtilis subspecies subtilis (Ehrenberg) Nakamura et al. strain DSM 1090, OUT 8424, Bacillus subtilis subspecies subtilis (Ehrenberg) Nakamura et al. strain DSM 1091, OUT 8425, Bacillus subtilis (Ehrenberg) Cohn strain DSM 1092, IFO 3009, NBRC 3009, OUT 8235, Bacillus subtilis subspecies subtilis (Ehrenberg) Nakamura et al. strain DSM 3256, IAM 1213, Bacillus subtilis subspecies subtilis (Ehrenberg) Nakamura et al. strain DSM 3257, IAM 1259, Bacillus subtilis (Ehrenberg) Cohn strain DSM 3258, IAM 1260, Bacillus subtilis (Ehrenberg) Cohn strain DSM 4181, NCA 72-52, SA 22, Bacillus subtilis subspecies subtilis (Ehrenberg) Nakamura et al. strain DSM 4393, pC194, SB202, Bacillus subtilis (Ehrenberg) Cohn strain DSM 4449, natto 3335 UM4, Bacillus subtilis (Ehrenberg) Cohn strain DSM 4450, natto 3335 UM8, pLS20, pBC16, Bacillus subtilis (Ehrenberg) Cohn strain DSM 4451, Bacillus subtilis (Ehrenberg) Cohn strain DSM 4515, DB163, pGR71, Bacillus subtilis (Ehrenberg) Cohn strain DSM 4608, BR157, pMW1, Bacillus subtilis subspecies subtilis (Ehrenberg) Nakamura et al. strain DSM 4750, 1E7, BGSC 1E7, pE194-cop6, Bacillus subtilis subspecies subtilis (Ehrenberg) Nakamura et al. strain DSM 4751, 1E34, BGSC 1E34, pAM77, Bacillus subtilis subspecies subtilis (Ehrenberg) Nakamura et al. strain DSM 4871, BD426, BGSC 1E21, pBD8, Bacillus subtilis subspecies subtilis (Ehrenberg) Nakamura et al. strain DSM 4872, BD466, BGSC 1E24, pBD10, Bacillus subtilis subspecies subtilis (Ehrenberg) Nakamura et al. strain DSM 4874, BGSC 1E38, pMK3, YB886, Bacillus subtilis subspecies subtilis (Ehrenberg) Nakamura et al. strain DSM 5213, BGSC 1A40, BR 151, Bacillus subtilis subspecies subtilis (Ehrenberg) Nakamura et al. strain DSM 5214, BD 393, BGSC 1A511, Bacillus subtilis subspecies subtilis (Ehrenberg) Nakamura et al. strain DSM 5545, BGSC 1A459/SU+III, Bacillus subtilis subspecies subtilis (Ehrenberg) Nakamura et al. strain DSM 5547, Bacillus subtilis (Ehrenberg) Cohn strain DSM 5552, Bacillus subtilis (Ehrenberg) Cohn strain DSM 5611, NRRL B-360, Bacillus subtilis subspecies subtilis (Ehrenberg) Nakamura et al. strain DSM 5660, NRRL B-362, Bacillus subtilis subspecies spizizenii Nakamura et al. strain DSM 6395, BGSC 2A2, W23 2A2, WB 672, Bacillus subtilis (Ehrenberg) Cohn strain DSM 6397, BGSC 1A2, SB 491, Bacillus subtilis subspecies spizizenii Nakamura et al. strain DSM 6399, BGSC 2A1, SB 623, Bacillus subtilis subspecies spizizenii Nakamura et al. strain DSM 6405, BGSC 2A3, W23 SR, Bacillus subtilis subspecies subtilis (Ehrenberg) Nakamura et al. strain DSM 6887, BGSC 1A309, NP40, Bacillus subtilis subspecies subtilis (Ehrenberg) Nakamura et al. strain DSM 6889, 1A658, BGSC 1A658, DA 65 Bacillus subtilis subspecies spizizenii Nakamura et al. strain DSM 8439, CCM 2268, IAM 12021, Bacillus subtilis (Ehrenberg) Cohn strain DSM 13019, SSI MK1, Bacillus subtilis subspecies spizizenii Nakamura et al. strain DSM 15029, NRRL B-23049, Bacillus subtilis subspecies inaquosorum Rooney et al. strain DSM 21200, Bacillus subtilis (Ehrenberg) Cohn strain DSM 21393, Bacillus subtilis subspecies inaquosorum Rooney et al. strain DSM 22148, KCTC 13429, Bacillus subtilis (Ehrenberg) Cohn strain DSM 23521, Bacillus subtilis (Ehrenberg) Cohn strain DSM 23778, Bacillus subtilis (Ehrenberg) Cohn strain DSM 25152, Bacillus subtilis (Ehrenberg) Cohn strain DSM 28592, Bacillus subtilis (Ehrenberg) Cohn strain DSM 30512, Bacillus subtilis (Ehrenberg) Cohn strain DSM 30529, Bacillus subtilis (Ehrenberg) Cohn strain DSM 30533, Bacillus subtilis (Ehrenberg) Cohn strain DSM 30534, Bacillus subtilis (Ehrenberg) Cohn strain DSM 30540, Bacillus subtilis (Ehrenberg) Cohn strain DSM 30541, Bacillus subtilis (Ehrenberg) Cohn strain DSM 30551, Bacillus subtilis (Ehrenberg) Cohn strain DSM 30558, Bacillus subtilis (Ehrenberg) Cohn strain DSM 30562, Bacillus subtilis (Ehrenberg) Cohn strain DSM 30570, Bacillus subtilis (Ehrenberg) Cohn strain DSM 30581, Bacillus subtilis (Ehrenberg) Cohn strain DSM 30597, Bacillus subtilis (Ehrenberg) Cohn strain DSM 30642, Bacillus subtilis (Ehrenberg) Cohn strain DSM 30651, Bacillus subtilis (Ehrenberg) Cohn strain DSM 30652, Bacillus subtilis (Ehrenberg) Cohn strain DSM 30671, Bacillus subtilis (Ehrenberg) Cohn strain DSM 30676, Bacillus subtilis (Ehrenberg) Cohn strain DSM 30677, Bacillus subtilis (Ehrenberg) Cohn strain DSM 30682, Bacillus subtilis (Ehrenberg) Cohn strain DSM 30711, Bacillus subtilis (Ehrenberg) Cohn strain DSM 30723, Bacillus subtilis (Ehrenberg) Cohn strain DSM 30801, Bacillus subtilis (Ehrenberg) Cohn strain DSM 30924, Bacillus subtilis (Ehrenberg) Cohn strain DSM 30925, Bacillus subtilis (Ehrenberg) Cohn strain DSM 30927, Bacillus subtilis (Ehrenberg) Cohn strain DSM 30928, Bacillus subtilis (Ehrenberg) Cohn strain DSM 30929, Bacillus subtilis (Ehrenberg) Cohn strain DSM 30941, D1, Bacillus subtilis (Ehrenberg) Cohn strain DSM 30942, D-FC1, Bacillus subtilis (Ehrenberg) Cohn strain DSM 31008, Bacillus subtilis (Ehrenberg) Cohn strain DSM 31009, Bacillus subtilis (Ehrenberg) Cohn strain DSM 31010, Bacillus subtilis (Ehrenberg) Cohn strain DSM 31020, Bacillus subtilis (Ehrenberg) Cohn strain DSM 31021, Bacillus subtilis (Ehrenberg) Cohn strain DSM 31033, Bacillus subtilis (Ehrenberg) Cohn strain DSM 100605, Bacillus subtilis (Ehrenberg) Cohn strain DSM 100612, Bacillus subtilis (Ehrenberg) Cohn strain DSM 100613, Bacillus subtilis (Ehrenberg) Cohn strain DSM 100614, Bacillus subtilis (Ehrenberg) Cohn strain DSM 103044, Bacillus subtilis (Ehrenberg) Cohn strain DSM 103047, Bacillus subtilis (Ehrenberg) Cohn strain DSM 103051, Bacillus subtilis (Ehrenberg) Cohn strain DSM 103758, Bacillus subtilis AM0904 (NRRL Deposit Number B-50914), Bacillus subtilis AM0911 (NRRL Deposit Number B-50915), Bacillus subtilis NP122 (NRRL Deposit Number B-50910), Bacillus subtilis NP119B (NRRL Deposit Number B-50909), Bacillus subtilis BS18 (NRRL B-50633), Bacillus subtilis BS278 (NRRL 50634), Bacillus subtilis 4-7d (NRRL B-50505), Bacillus subtilis 3-5h (NRRL B-50507), Bacillus subtilis AGTP BS3BP5 (NRRL B-50510), Bacillus subtilis BS918 (NRRL B-50508), Bacillus subtilis AGTP BS1013 (NRRL-50509), Bacillus subtilis AGTP 944 (NRRL B-50548), Bacillus subtilis AGTP BS442 (NRRL B-50542), Bacillus subtilis AGTP BS1069 (NRRL B-50544), Bacillus subtilis AGTP BS521 (NRRL B-50545), Bacillus subtilis B27 (NRRL B-50105), Bacillus subtilis 3A-P4 (PTA-6506), Bacillus subtilis 22C-P1 (PTA-6508), Bacillus subtilis BL21 (NRRL B-50134), Bacillus subtilis strain GB03, Bacillus subtilis strain QST713, Bacillus subtilis DSM 5750 (BioPlus® 2B, Chr. Hansen Bio Systems), and Bacillus subtilis PR104 (NRRL-B-68096).

### IV. Amounts of Bacillus licheniformis, Bacillus Pumilus, and Bacillus subtilis in Bacilli Combinations

The relative amounts of Bacillus licheniformis, Bacillus pumilus, and Bacillus subtilis present in the Bacilli combination are selected to obtain a desired result. For certain aspects, the Bacilli combination comprises from about 10² to about 10¹² CFU/gram, and more typically from about 10² to about 10¹¹ CFU/gram or from about 10³ to 10¹⁰ CFU/gram of each of the Bacillus species in the Bacilli combination. In some aspects, the Bacilli combination comprises from about 10⁴ to about 10⁶ CFU/gram of each of the Bacillus species in the Bacilli combination, and/or may include from about 10⁵ to about 10⁷ CFU/gram of the Bacilli combination in total per gram of feed.

In some aspects, the Bacilli combination may be formulated and/or administered to provide different CFU ratios of each Bacillus species included therein. For example, one aspect includes, per gram of finished feed, 10⁵ CFU of Bacillus licheniformis, 10⁵ of Bacillus pumilus, and 10⁵ CFU of Bacillus subtilis.

For another example, an aspect can include per gram of finished feed, 1 to 5×10⁵ CFU of Bacillus licheniformis, 1 to 5×10⁵ CFU of Bacillus pumilus, and 1 to 5×10⁵ CFU of Bacillus subtilis for a final total concentration of approximately 10⁶ CFU of Bacilli species per gram of finished food. In other aspects, the formulation can include per gram of finished feed, from 1 to 5×10⁵ CFU of Bacillus licheniformis, from 1 to 5×10⁵ CFU of Bacillus pumilus, and from 2 to 8×10⁵ CFU of Bacillus subtilis for a final total concentration of approximately 10⁶ CFU of Bacilli species per gram of finished food.

For another example, an aspect can include per gram of finished feed, 3×10⁵ CFU of Bacillus licheniformis, 2×10⁵ of Bacillus pumilus, and 5×10⁵ CFU of Bacillus subtilis for a final total concentration of 1×10⁶ CFU per gram of finished food.

Another aspect can include per gram of finished feed, 5×10⁵ CFU of Bacillus licheniformis, 2×10⁵ of Bacillus pumilus, and 3×10⁵ CFU of Bacillus subtilis for a final total concentration of 1×10⁶ CFU per gram of finished food.

Another aspect can include per gram of finished feed, 2×10⁵ CFU of Bacillus licheniformis, 3×10⁵ of Bacillus pumilus, and 5×10⁵ CFU of Bacillus subtilis for a final total concentration of 1×10⁶ CFU per gram of finished food.

Another aspect can include per gram of finished feed, 2×10⁵ CFU of Bacillus licheniformis, 5×10⁵ of Bacillus pumilus, and 3×10⁵ CFU of Bacillus subtilis for a final total concentration of 1×10⁶ CFU per gram of finished food.

Another aspect can include per gram of finished feed, 5×10⁵ CFU of Bacillus licheniformis, 3×10⁵ of Bacillus pumilus, and 2×10⁵ CFU of Bacillus subtilis for a final total concentration of 1×10⁶ CFU per gram of finished food.

Another aspect can include per gram of finished feed, 3×10⁵ CFU of Bacillus licheniformis, 2×10⁵ of Bacillus pumilus, and 5×10⁵ CFU of Bacillus subtilis for a final total concentration of 1×10⁶ CFU per gram of finished food.

Another aspect can include per gram of finished feed, 3×10⁶ CFU of Bacillus licheniformis, 2×10⁶ of Bacillus pumilus, and 5×10⁶ CFU of Bacillus subtilis for a final total concentration of 1×10⁷ CFU per gram of finished food.

Another aspect can include per gram of finished feed, 5×10⁶ CFU of Bacillus licheniformis, 2×10⁶ of Bacillus pumilus, and 3×10⁶ CFU of Bacillus subtilis for a final total concentration of 1×10⁷ CFU per gram of finished food.

Another aspect can include per gram of finished feed, 2×10⁶ CFU of Bacillus licheniformis, 3×10⁶ of Bacillus pumilus, and 5×10⁶ CFU of Bacillus subtilis for a final total concentration of 1×10⁷ CFU per gram of finished food.

Another aspect can include per gram of finished feed, 2×10⁶ CFU of Bacillus licheniformis, 5×10⁶ of Bacillus pumilus, and 3×10⁶ CFU of Bacillus subtilis for a final total concentration of 1×10⁷ CFU per gram of finished food.

Another aspect can include per gram of finished feed, 5×10⁶ CFU of Bacillus licheniformis, 3×10⁶ of Bacillus pumilus, and 2×10⁶ CFU of Bacillus subtilis for a final total concentration of 1×10⁷ CFU per gram of finished food.

Another aspect can include per gram of finished feed, 3×10⁶ CFU of Bacillus licheniformis, 2×10⁶ of Bacillus pumilus, and 5×10⁶ CFU of Bacillus subtilis for a final total concentration of 1×10⁷ CFU per gram of finished food.

In some aspects, the Bacilli combination may be formulated and/or administered to provide different concentrations or percentages of each Bacillus species included therein. For example, with respect to other Bacillus species in the Bacilli combination, the total amount of Bacillus subtilis relative to the other Bacillus species may be from greater than zero to 99%, such as from 10% to 90%, from 15% to 85%, from 20% to 80%, from 25% to 75%, from 35% to 65%, from 45% to 55%, or substantially 50%, such that the total percentage of all Bacilli species is 100%. For example, the total amount of Bacillus licheniformis relative to the other Bacillus species may be from greater than zero to 99%, such as from 10% to 90%, from 15% to 85%, from 20% to 80%, from 25% to 75%, from 35% to 65%, from 45% to 55%, from 10% to 50%, from 10% to 40%, from 15% to 30%, substantially 20% or substantially 50%, such that the total percentage of all Bacilli species is 100%. For example, the total amount of Bacillus pumilus relative to the other Bacillus species may be from greater than zero to 99%, such as from 10% to 90%, from 15% to 85%, from 20% to 80%, from 25% to 75%, from 35% to 65%, from 45% to 55%, from 10% to 50%, from 15% to 40%, from 20% to 40%, substantially 30% or substantially 50%, such that the total percentage of all Bacilli species is 100%. In one aspect, the total amount of B. subtilis relative to the other Bacillus species is about 50%, the total amount of B. licheniformis relative to the other Bacillus species is about 30%, and the total amount of B. pumilus relative to the other Bacillus species is about 20%, such that the total percentage of all Bacilli species is 100%.

In another aspect, the total amount of B. subtilis relative to the other Bacillus species is about 50%, the total amount of B. licheniformis relative to the other Bacillus species is about 20%, and the total amount of B. pumilus relative to the other Bacillus species is about 30%, such that the total percentage of all Bacilli species is 100%.

### V. Additional Component(s)

The Bacilli combination also can be administered in combination with one or more additional components or compositions. An additional component or composition may be any component or composition that can be administered to a subject, particularly an animal, such as an avian, including poultry, in combination with the Bacilli species in the Bacilli combination. Certain disclosed Bacilli combinations are particularly formulated for administration to poultry, and therefore can comprise the Bacilli combination in combination with any other component or composition now known or hereafter developed for administration to poultry. Exemplary additional components include a carrier, a vitamin, a copper salt, allicin, alliin, alliinase, algae, a polyphenol or plant material comprising polyphenol, a feed supplement (for example, yucca, quillaja, silica, mineral clay, glucan, mannans, and/or an additional DFM), a feed (for example, a poultry feed, ruminant feed, or aquatic species feed), or a combination thereof. Typically, if the additional component comprises an additional DFM, such additional DFM is not a Bacillus species. The additional component(s) will comprise from 1 wt % to 99 wt % and the Bacilli combination will comprise from 99 wt % to 1 wt % of the total weight of the combination. In some aspects, the additional component(s) will comprise from 10 wt % to 90 wt % and the Bacilli combination will comprise from 90 wt % to 10 wt % of the total weight of the combination. In some aspects, the additional component(s) will comprise 20 wt % to 80 wt % and the Bacilli combination will comprise from 80 wt % to 20 wt % of the total weight of the combination. The Bacilli combination may be administered with the other component(s), optionally in a mixture with the other component(s), such as poultry feed and/or a feed supplement, in an amount sufficient to provide the desired amounts of the respective Bacillus species in the particular combination.

### A. Carrier

In some aspects, the Bacilli combination may be mixed with and/or dispersed in a carrier to form a dispersed composition. The carrier(s) may be selected to provide a non-biological benefit to the composition, compared to a Bacilli combination without a carrier, such as, but not limited to, achieving or improving a readily flowable state, and/or improving stability during storage and/or transport. Suitable carriers that may be used in combination with a Bacilli combination include, but are not limited to, plant material, such as beet pulp, ground corn, corn syrup solids, plant fiber, rice hulls, soluble plant fiber, wheat middlings, microcrystalline cellulose; carbonates, such as metal carbonates, such as calcium carbonate, potassium carbonate; sulfates, such as metal sulfates, such as potassium sulfate, sodium sulfate; lactates, including metal lactates, such as calcium lactate; oxides, including metal oxides, such as calcium oxide; propionates, including metal propionates, such as calcium propionate; stearates, including metal stearates, such as calcium stearate; phosphates, such as dicalcium phosphate dehydrate, monocalcium phosphate, sodium tripolyphosphate, or tetra sodium pyrophosphate; minerals, such as dolomite, silicon dioxide, silica, limestone, or vermiculite; clays, such as bentonite, montmorillonite, kaolin; sugars, such as glucose, sucrose, dextrose, fructose, or a combination thereof; maltodextrin; salt, such as sodium chloride; carrageenan; cellulose; guar gum; polyols; sodium alumino silicate; urea; animal protein products; forage products; grain products; plant protein products; processed grain products; roughage products; molasses products; oil, such as mineral oil, vegetable oil, corn oil, soybean oil, or a combination thereof; or combinations thereof. In some aspects, the carrier is or comprises calcium carbonate. In particular aspects, the carrier is or comprises calcium carbonate, mineral oil, vegetable oil, dextrose, maltodextrin, or any combination thereof.

Animal protein products may include, but are not limited to, blood meal; animal by-product meal; buttermilk, including condensed buttermilk and dried buttermilk; casein; dried hydrolyzed casein; cheese rind; crab meal; fish products, including fish by-products, fish liver and glandular meal, fish meal, fish protein concentrates, fish residue meal, and dried and/or condensed fish solubles; fleshings hydrolysate; hydrolyzed hair; hydrolyzed leather meal; hydrolyzed poultry by-product aggregate; hydrolyzed poultry feathers; leather hydrolysate; meat and bone meal; meat and bone meal tankage; meat meal; meat meal tankage; dried meat solubles; dried lactalbumin; dried feed grade milk; dried milk protein; poultry by-products and/or by-products meal; poultry hatchery by-product; shrimp meal; skimmed milk, including condensed, condensed cultured, dried, or dried cultured skimmed milk; whey, including condensed, condensed cultured, condensed hydrolyzed, dried, or dried hydrolyzed whey; condensed and/or dried whey product; condensed and/or dried whey solubles; or a combination thereof.

Forage products may include, but are not limited to, alfalfa products, such as dehydrated meal, optionally in pellet form, ground hay, or suncured meal, optionally in pellet form; coastal bermudagrass hay; dehydrated corn plant; dehydrated silage; flax plant product; ground grass; lespedeza meal and/or stem meal; ground soybean hay; or combinations thereof.

Grain products may include, but are not limited to, barley, corn, grain sorghum, mixed feed oats, oats, triticale, wheat, ground brown rice, ground or ground paddy rough rice, broken or chipped rice, brewers rice, rye, or a combination thereof. The grain products may be in any suitable form, such as whole, ground, cracked, screen cracked, flaked, kibbled, toasted, and/or heat processed.

Plant protein products may include, but are not limited to, dried beans; canola meal; coconut meal; cottonseed, such as flakes, cake, meal, low gossypol meal, and/or whole pressed cottonseed; guar meal; dried kelp; linseed meal; peanut meal; peas; potato protein; dried seaweed meal; safflower meal; soy protein concentrate; soybean feed; ground soybeans; soybean meal, optionally kibbled; heat processed soybeans; ground, extruded whole soybeans; soy flour; soy grits; sunflower meal, optionally dehulled; yeast, such as active dried yeast, brewers dried yeast, culture yeast, dried yeast, primary dried yeast, torula dried yeast, and/or candida dried yeast; or a combination thereof.

The processed grain by-products may be aspirated grain fractions; brewers dried grains; buckwheat middlings; condensed distillers solubles; condensed fermented corn extracts; corn bran; corn flour; corn germ meal; corn gluten feed and/or meal; corn grits; distillers dried grains, optionally with solubles; distillers dried solubles, flour, grain sorghum germ cake, meal, grits, and/or mill feed; meal hominy feed; malt sprouts; oat groats; feeding oat meal; pearl barley by-product; peanut skins; rice bran; rice polishings; rye middlings; gelatinized or partially aspirated sorghum grain flour; wheat bran, flour, shorts, germ meal, defatted germ meal, middlings, mill run and/or red dog; or a combination thereof.

Roughage products may include, but are not limited to, almond hulls; dried apple pectin pulp; dried apple pomace; bagasse; barley hulls; barley mill by-product; dried, plain beet pulp; buckwheat hulls; dried citrus meal; dried citrus pulp; citrus seed meal; corn cob fractions; cottonseed hulls; flax straw by-product; ground corn cob; psyllium seed husk; malt hulls; clipped oat by-product; oat hulls; oat mill by-product; peanut hulls; rice hulls; rice mill by-product; rye mill run; soybean hulls, mill feed, and/or mill run; sunflower hulls; ground straw; dried tomato pomace; or a combination thereof.

Molasses products may be beet molasses; dried beet molasses product; dried beet pulp molasses; cane molasses; citrus molasses; molasses yeast condensed solubles; concentrated separator by-product; condensed molasses fermentation solubles; starch molasses; molasses distillers condensed solubles; molasses distillers dried solubles; or a combination thereof.

### B. Copper Species

The disclosed combination may be mixed with a copper species such as a copper species that provides a copper ion. The copper species may be a copper salt. Exemplary copper species that may be combined with the Bacilli combination include, but are not limited to, copper chloride, copper bromide, copper iodide, copper sulfate, copper sulfite, copper bisulfite, copper thiosulfate, copper phosphate, monobasic copper phosphate, dibasic copper phosphate, copper hypophosphite, copper dihydrogen pyrophosphate, copper tetraborate, copper borate, copper carbonate, copper bicarbonate, copper metasilicate, copper citrate, copper malate, copper methionate, copper succinate, copper lactate, copper formate, copper acetate, copper butyrate, copper propionate, copper benzoate, copper tartrate, copper ascorbate, copper gluconate, or a combination thereof. In certain aspects, the copper salt is copper chloride, copper sulfate, copper phosphate, copper oxide, copper glycinate, copper hydroxide, basic copper chloride, copper dihydrogen pyrophosphate, copper carbonate, copper citrate, copper acetate, copper gluconate, or a combination thereof, such as basic copper chloride, copper carbonate, copper glycinate, copper oxide, copper sulfate, or a combination thereof. A copper species, such as a copper salt, may be provided separately, or individually, or it may be provided as part of a composition, such as a feed or a feed supplement. Certain disclosed aspects comprise, consist essentially of, or consist of Bacillus lichemformis, Bacillus pumilis, Bacillus subtilis, and a copper species. In any aspects, the copper species may be a copper salt, such as a salt that can provide a copper ion, for example, copper sulfate.

### C. Vitamin(s)

The disclosed combination may be mixed with a vitamin or vitamins. Exemplary vitamins include, but are not limited to, one or more of Vitamin A, Vitamin B₁ (thiamine), Vitamin B₂ (riboflavin), Vitamin B₃ (niacin or niacinamide), Vitamin B₅ (pantothenic acid), Vitamin B₆ (pyridoxine, pyridoxal, or pyridoxamine, or pyridoxine hydrochloride), Vitamin B₇ (biotin), Vitamin B₉ (including folic acid), Vitamin B₁₂ (various cobalamins; commonly cyanocobalamin in vitamin supplements), Vitamin C (ascorbic acid or a salt thereof, such as sodium ascorbate or calcium sorbate), Vitamin D (vitamin D₁, vitamin D₂, vitamin D₃, vitamin D₄, vitamin D₅, 25-hydroxy vitamin D₃, 25-dihydroxy vitamin D₃, or combinations thereof), Vitamin E, Vitamin K (K1 and K2 (i.e., MK-4, MK-7)), and biotin, and derivatives, salts and/or analogs thereof.

### D. Feed

The feed may be any feed suitable for administration to an animal. The Bacilli combination may be administered in combination with the feed, such as by forming a mixture of the Bacilli combination and the feed, or by administering the Bacilli combination and the feed sequentially, in any order. In certain disclosed aspects the animal is a poultry, and the Bacilli combination is used in combination with, and may be admixed with, a poultry feed, such as a poultry basal diet. The feed may comprise corn, alfalfa, peas, soybean meal, soybean oil, wheat, oats, sorghurn, barley, rye, rice hulls, canola, corn oil, limestone, salt (for example, sodium chloride), distillers dried grains with solubles (DDGS), dicalcium phosphate, sodium sesquicarbonate, methionine source, lysine source, L-threonine, mineral oil, biotin, folic acid, kelp, menadione dimethylpyrimidinol bisulfite, calcium aluminosilicate, or any combination thereof. The feed may also comprise one or more additional components. Additional components may be used for any desired purpose, such as a substantially biologically inert material added, for example, as a filler, or to provide a desired beneficial effect. For example, the feed may include a carbonate (including a metal carbonate such as calcium carbonate); a trace mineral, such as, but not limited to, chloride, fluoride, iodide, chromium, copper, zinc, iron, magnesium, manganese, molybdenum, phosphorus, potassium, sodium, sulfur, selenium, or a combination thereof; a bulking agent; a carrier; a colorant; a taste enhancer; a preservative; one or more vitamins; or a combination thereof. The preservative may be benzoic acid or a salt thereof, e.g. sodium benzoate; lactic acid or a salt thereof, e.g. sodium lactate, potassium lactate or calcium lactate; propionic acid or a salt thereof, e.g. sodium propionate; ascorbic acid or a salt thereof, e.g. sodium ascorbate; gallic acid or a salt thereof e.g. sodium gallate; sulfur dioxide and/or sulfites; nitrites; nitrates; choline, or a salt thereof, such as an anion salt of choline, e.g. choline halide, such as chloride, bromide, iodide, fluoride, or choline hydroxide; or any combination thereof. The one or more vitamins may include vitamin A; vitamin B₁, such as thiamine mononitrate; vitamin B₂, such as riboflavin-5-phosphate; vitamin B₃, such as niacin or niacinamide; vitamin B₅, such as pantothenic acid or d-calcium pantothenate; vitamin B₆, such as pyridoxine or pyridoxine hydrochloride; vitamin B₁₂; vitamin C, such as ascorbic acid, sodium ascorbate, or calcium sorbate; vitamin D; vitamin E; vitamin K, or a combination thereof. Vitamin D may comprise vitamin D₁, vitamin D₂, vitamin D₃, vitamin D₄, vitamin D₅, 25-hydroxy vitamin D₃, 25-dihydroxy vitamin D₃, or combinations thereof.

The feed, such as a poultry feed, may also include fats and/or oils, such as tallow, optionally derived from the rendering of beef offal; lard, optionally derived from the rendering of pork offal; poultry fat, optionally derived from poultry offal; feed grade animal fat, optionally derived from a mixture of rendered beef, pork, and/or poultry raw material; yellow grease, optionally derived from reprocessed restaurant grease and/or cooking oil; and/or blended animal-vegetable fat, which may include blends of different types and/or amounts of animal fats and vegetable oils from restaurant grease. Additionally, or alternatively, the feed may include protein sources, such as canola, fish meal, field peas, meat and bone meal, soybeans, and/or cereal by-products.

E. Allicin, Alliin, and/or Alliinase

Allicin (diallyl thiosulfate; 2-Propene-1-sulfinothioic acid S-2-propenyl ester) is a compound found in garlic, such as raw garlic. Allicin is typically produced from alliin ((2R)-2-amino-3-[(S)-prop-2-enylsulfinyl]propanoic acid) in damaged garlic cells by the action of the enzyme alliinase. Allicin, alliin, and/or alliinase may be provided as whole garlic cloves or bulbs; crushed, mashed, or chopped garlic; a garlic extract; and/or as a synthesized or isolated compound.

### F. A Polyphenol Compound

The polyphenol may be provided by a plant extract from a polyphenol-containing plant material. The plant material also may include non-polyphenol compounds, including polyphenol degradation products, such as gallic acid and trans-caftaric acid. Degradation can occur, for example, through oxidative and/or biological processes. Both the polyphenols and the non-polyphenol compounds may have biological activity. The plant extract may be prepared from a single plant material or from a combination of plant materials. Suitable plant materials from which a plant extract can be obtained include, but are not limited to, apples, blackberries, black chokeberries, black currants, black elderberries, blueberries, cherries, cranberries, grapes, green tea, hops, onions, quillaja, plums, pomegranates, raspberries, strawberries, and yucca.

In some aspects, the plant extract is prepared from a pressed plant material, such as grape pomace, a dried plant material, such as tea, or a combination thereof. Pomace may be obtained substantially immediately post-pressing or as an ensiled product, i.e., pomace collected and stored for up to several months post-pressing. Suitable plants have a plurality of polyphenols and/or other non-polyphenolic compounds including, but not limited to, non-polyphenolic organic acids (such as gallic acid and/or trans-caftaric acid), flavanols, gallate esters, flavanodiols, phloroglucinol, pyrogallol, and catechol. In some aspects, the plant extract is prepared from Pinot noir pomace, Pinot gris pomace, or green tea.

In some aspects, pressed or dried plant material is ground to a fine powder prior to, or during, extraction. Pressed plant materials may be frozen to facilitate grinding. Polyphenols and other non-polyphenolic compounds may be extracted for administration. For example, polyphenols and other non-polyphenolic compounds may be extracted from the powder using a solution comprising a polar solvent, such as water, an alcohol, an ester, or a combination thereof. In some aspects, the solution comprises a water-miscible alcohol, ester, or combination thereof, such as a lower alkyl alcohol, lower alkyl ester, or a combination thereof. In some aspects, the solution is water or an aqueous solution comprising 25-99% solvent, such as 25-95% solvent, 30-80% solvent, or 50-75% solvent, and water. In certain aspects, the solution is an aqueous solution comprising methanol, ethanol, isopropanol, ethyl acetate, or a combination thereof. The solution may be acidified by addition of an acid. The acid may prevent or minimize oxidative degradation of biologically-active polyphenols and other non-polyphenolic compounds in the extract. The acid may be any suitable acid, such as a mineral acid (e.g., hydrochloric acid), or an organic acid such as citric acid or acetic acid. In some aspects, the solution comprises from 0.01% to 1% acid, such as 0.02-0.5%, 0.025-0.25%, or 0.05-0.15%. In some examples, the solution includes 0.1% hydrochloric acid.

Extraction may be performed at a temperature ranging from 0-100° C. In some aspects, extraction is performed at a temperature ranging from 20-70° C., or at ambient temperature. Extraction may be performed for a duration ranging from several minutes to several days. To increase extraction efficiency, the plant material and solution may be mixed or agitated during extraction, such as by grinding the plant material during extraction, stirring the mixture, shaking the mixture, or homogenizing the mixture. In some aspects, the extraction may be repeated one or more times with fresh solution to increase recovery of polyphenols and other non-polyphenolic compounds from the plant material. The liquid phases from each extraction cycle are then combined for further processing.

The liquid phase can be recovered, and the residual solids, or pulp, are discarded. Recovering the liquid phase may comprise decanting the liquid from the remaining solids and/or filtering the liquid phase to remove residual solids. The solvent (alcohol, ester, or combination thereof) can be removed from the liquid solution by any suitable means, such as evaporation (e.g., roto-evaporation), to produce an aqueous extract containing the biologically-active components in a mildly acidic solution.

In certain aspects where the plant material includes a significant amount of oils, or lipids, an initial extraction of nonpolar components may be performed before extracting the polyphenols and other polar, non-polyphenolic compounds. Nonpolar components may be extracted by homogenizing the plant material in a nonpolar solvent, e.g., hexanes, heptanes, or a combination thereof. The solvent layer including the extracted nonpolar components is separated from the plant material and discarded.

The aqueous plant extract may be further purified by suitable means, e.g., extraction, chromatographic methods, distillation, etc., to remove non-polyphenolic compounds and/or to increase the concentration of polyphenols relative to other compounds in the extract.

The aqueous plant extract may be dried, for example by freeze-drying or other low-temperature drying methods, and ground to a powder to provide a dried plant extract. In some aspects, the dried plant extract comprises 0.01 wt % to 25 wt % total polyphenols, such as 0.01 wt % to 10 wt %, 0.01 wt % to 5 wt %, 0.01 wt % to 2.5 wt %, 0.01 wt % to 1 wt %, 0.01 wt % to 0.5 wt %, 0.02 to 0.25 wt %, or 0.03-0.1 wt % total polyphenols. In certain aspects, the dried plant extract further comprises non-polyphenolic compounds. For example, the dried plant extract may comprise 0.01-1 mg/g gallic acid, such as 0.05-0.5 mg/g or 0.09-0.25 mg/g gallic acid, and/or 0.001-0.1 mg/g trans-caftaric acid, such as 0.005-0.05 mg/g or 0.01-0.025 mg/g trans-caftaric acid.

The aqueous plant extract may be concentrated to a smaller volume, e.g., by evaporation, and used as an aqueous plant extract. In other aspects, the aqueous plant extract is mixed with a carrier before drying and grinding. Suitable carriers include, for example, diatomaceous earth, silica, maltodextrin, ground grain (e.g., corn), meals (e.g., soybean or cottonseed meal) by-products (e.g., distiller's dried grains, rice hulls, wheat mill run), clays (e.g., bentonite), and combination thereof. The plant extract may be combined with a carrier in a ratio ranging from 10:1 to 1:10 by weight, such as from 5:1 to 1:5. For example, the plant extract may be mixed with diatomaceous earth in a ratio of 3:1 by weight.

### G. Feed Supplements

The Bacilli combination may be used in combination with one or more feed supplements. In some aspects, the Bacilli combination is mixed with the feed supplement to form a mixture or composition comprising the Bacilli combination and the feed supplement(s). In other aspects, the Bacilli combination is administered in combination with a feed supplement.

1. Yucca and/or Quillaja, or Extracts Thereof

A disclosed Bacilli combination may be administered in combination with yucca and/or quillaja plant material, or extracts thereof. Examples of yucca include, but are not limited to, Yucca aloifolia, Yucca angustissima, Yucca arkansana, Yucca baccata, Yucca baileyi, Yucca brevifolia, Yucca campestris, Yucca capensis, Yucca carnerosana, Yucca cernua, Yucca coahuilensis, Yucca constricta, Yucca decipiens, Yucca declinata, Yucca de-smetiana, Yucca elata, Yucca endlichiana, Yucca faxoniana, Yucca filamentosa, Yucca filifera, Yucca flaccida, Yucca gigantean, Yucca glauca, Yucca gloriosa, Yucca grandiflora, Yucca harrimaniae, Yucca intermedia, Yucca jaliscensis, Yucca lacandonica, Yucca linearifolia, Yucca luminosa, Yucca madrensis, Yucca mixtecana, Yucca necopina, Yucca neomexicana, Yucca pallida, Yucca periculosa, Yucca potosina, Yucca queretaroensis, Yucca reverchonii, Yucca rostrata, Yucca rupicola, Yucca schidigera, Yucca schottii, Yucca sterilis, Yucca tenuistyla, Yucca thompsoniana, Yucca treculeana, Yucca utahensis, Yucca valida or combinations thereof. In certain aspects, the Yucca is or comprises Yucca schidigera.

Examples of quillaja include, but are not limited to, Quillaja brasiliensis, Quillaja lanceolata, Quillaja lancifolia, Quillaja molinae, Quillaja petiolaris, Quillaja poeppigii, Quillaja saponaria, Quillaja sellowiana, Quillaja smegmadermos or combinations thereof. In certain aspects, the quillaja is or comprises Quillaja saponaria.

A person of ordinary skill in the art will appreciate that, as used herein, a plant name may refer to the plant as a whole, or to any part of the plant, such as the roots, stem or trunk, bark, leaves, flower, flower stems, seeds, or a combination thereof. These plant parts may be used fresh, or dried, and may be whole, pulverized, or comminuted. The plant name may also refer to extracts from any part or parts of the plant, such as chemical extracts, or extracts obtained by pressing, or any other methods of concentrating or extracting oils or other extracts known to those in the art or that are hereafter discovered. Plant extracts may include compounds that are saponins, triterpenoids, polyphenols, antioxidants or resveratrol, or combinations thereof.

The combination may comprise a composition comprising yucca and/or quillaja that may also include carriers and binding agents suitable to formulate the yucca and/or quillaja for administration to an animal. In certain aspects, such a composition can be a commercially available product, such as a composition comprising Yucca schidigera and Quillaja saponaria, sold under the trademark NUTRAFITO PLUS™ by Desert King International and/or MAGNI-PHI® by Phibro Animal Health Corporation. Such compositions may comprise from 99% or more Quillaja saponaria and 1% or less Yucca schidigera to 75% Quillaja saponaria and 25% Yucca schidigera, such as from 95% Quillaja saponaria and 5% Yucca schidigera to 80% Quillaja saponaria and 20% Yucca schidigera, and in certain aspects, 85% Quillaja saponaria and 15% Yucca schidigera, or from 90% to 95% Quillaja saponaria and from 5% to 10% Yucca schidigera, such as from 92% to 93% Quillaja saponaria and from 7% to 8% Yucca schidigera, or about 92.5% Quillaja saponaria and about 7.5% Yucca schidigera.

### 2. Silica, Mineral Clay, Glucan and Mannans

Additionally, or alternatively, a Bacilli combination can be administered in combination with a feed supplement comprising silica, mineral clay, glucan and mannans. The feed supplement may further comprise an endoglucanohydrolase, either endogenously or as an affirmatively added ingredient. As used herein, weight % for endoglucanohydrolase is based on a 70,000 unit/gram endoglucanohydrolase product. The endoglucanohydrolase may be β-1,3 (4)-endoglucanohydrolase.

In any aspects disclosed herein, the feed supplement may comprise, consist essentially of, or consist of, glucan (e.g., β-1,3 (4)glucan), silica, mineral clay and mannans. In some aspects, the feed supplement comprises, consists essentially of, or consists of, glucan (e.g., β-1,3 (4)glucan), silica, mineral clay, mannans and endoglucanohydrolase. In any aspects disclosed herein, the glucan and mannans may be provided, at least in part, by yeast cell wall or an extract thereof. Thus, in some aspects, the feed supplement may comprise, consist essentially of, or consist of, silica, mineral clay and yeast cell wall or an extract thereof, or the feed supplement may comprise, consist essentially of, or consist of, silica, mineral clay, yeast cell wall or an extract thereof, and endoglucanohydrolase. Similarly, endoglucanohydrolase may, in certain disclosed aspects, be provided by yeast cell wall or a yeast cell wall extract.

Suitable sources of silica include, but are not limited to, sand, diatomaceous earth, and synthetic silica. In one aspect, quartz may be used. In certain aspects, the mannans comprise glucomannan.

The components of the feed supplement are prepared by methods commonly known in the art and can be obtained from commercial sources. β-1,3 (4)-endoglucanohydrolase may be produced from submerged fermentation of a strain of Trichoderma longibrachiatum. Diatomaceous earth is available as a commercially-available product with from 70% to 95% silica (SiO₂) and with its remaining components not assayed but primarily ash (minerals) as defined by the Association of Analytical Chemists (AOAC, 2002). The mineral clays (e.g., aluminosilicates) used in this feed supplement may be any of a variety of commercially-available clays including, but not limited to, montmorillonite clay, bentonite and zeolite. Glucan, mannans, and/or endoglucanohydrolase can be obtained from plant cell walls, yeast or yeast cell wall or an extract thereof (e.g., Saccharomyces cerevisiae, Candida utilis), certain fungi (e.g., mushrooms), algae, and bacteria. In certain aspects, yeast can be administered affirmatively to provide glucan, mannans and endoglucanohydrolase endogenously.

In one aspect, the feed supplement comprises, consists essentially of, or consists of, 1-40 wt % silica, 0.5-25 wt % glucan and mannans, and 40-92 wt % mineral clay, in amounts relative to each other. In another aspect, the feed supplement comprises, consists essentially of, or consists of, 5-40 wt % silica, 0.5-15 wt % glucan and mannans, and 40-80 wt % mineral clay, in amounts relative to each other. In another aspect, the feed supplement comprises, consists essentially of, or consists of, 20-40 wt % silica, 0.5-10 wt % glucan and mannans, and 50-70 wt % mineral clay, in amounts relative to each other. In another aspect, the feed supplement comprises, consists essentially of, or consists of, 15-40 wt % silica, greater than zero to 15 wt % glucans, greater than zero to 10 wt % mannans, and 50-81 wt % mineral clay, in amounts relative to each other. In another aspect, the feed supplement comprises, consists essentially of, or consists of, 15-40 wt % silica, 0.5-5.0 wt % glucans, 0.5-8.0 wt % mannans, and 50-81 wt % mineral clay, in amounts relative to each other. In another aspect, the feed supplement comprises, consists essentially of, or consists of, 20-30 wt % silica, 0.5-3.5 wt % glucans, 0.5-6.0 wt % mannans, and 60-70 wt % mineral clay, in amounts relative to each other.

In some aspects, β-glucans and mannans are obtained from yeast or yeast cell wall or an extract thereof. The feed supplement may comprise, consist essentially of, or consist of, 1-40 wt % silica, 1-30 wt % yeast cell wall or an extract thereof, and 40-92 wt % mineral clay, in amounts relative to each other. In one aspect, the feed supplement comprises, consists essentially of, or consists of, 10-40 wt % silica, 5-20 wt % yeast cell wall or an extract thereof, and 40-80 wt % mineral clay, in amounts relative to each other. In another aspect, the feed supplement comprises, consists essentially of, or consists of, 15-30 wt % silica, 5-15 wt % yeast cell wall or an extract thereof, and 50-70 wt % mineral clay, in amounts relative to each other.

In any of the above aspects, the feed supplement may further comprise an endoglucanohydrolase, such as 3-1,3 (4)-endoglucanohydrolase. The feed supplement may include from 0.025 wt % endoglucanohydrolase to 5 wt % endoglucanohydrolase or more, such as from 0.05 wt % to 3 wt % 3-1,3 (4)-endoglucanohydrolase, relative to the amounts of silica, mineral clay, glucan, mannans, and/or yeast, yeast cell wall, or yeast cell wall extract present in the feed supplement. In one aspect, the feed supplement comprises, consists essentially of, or consists of, 0.1-3 wt % 3-1,3 (4)-endoglucanohydrolase, 20-40 wt % silica, 0.5-20 wt % glucan and mannans, and 50-70 wt % mineral clay, in amounts relative to each other. In another aspect, the feed supplement comprises, consists essentially of, or consists of, 0.1-3 wt %, 3-1,3 (4)-endoglucanohydrolase, 20-40 wt % silica, 0.5-10 wt % glucan and mannans, and 50-70 wt % mineral clay, in amounts relative to each other. Alternatively, the feed supplement may comprise, consist essentially of, or consist of, 0.1-3 wt % 3-1,3 (4)-endoglucanohydrolase, 1-40 wt % silica, 5-30 wt % yeast cell wall or an extract thereof, and 40-92 wt % mineral clay, in amounts relative to each other. In one aspect, the feed supplement comprises, consists essentially of, or consists of, 0.1-3 wt % 3-1,3 (4)-endoglucanohydrolase, 10-40 wt % silica, 5-20 wt % yeast cell wall or an extract thereof, and 40-80 wt % mineral clay, in amounts relative to each other. In another aspect, the feed supplement comprises, consists essentially of, or consists of, 0.1-3 wt % β-1,3 (4)-endoglucanohydrolase, 15-30 wt % silica, 5-15 wt % yeast cell wall or an extract thereof, and 50-70 wt % mineral clay, in amounts relative to each other.

In any of the above aspects, the silica may be provided by diatomaceous earth. In any of the above aspects, the glucans may be β-glucans. In some aspects, the β-glucans can be obtained from yeast, or other materials, such as fungi, algae, bacteria, or the like. In any of the above aspects, the mannans may comprise glucomannan.

The glucan and mannans (or yeast or yeast cell wall or an extract thereof) can be prepared by a method known to a person of ordinary skill in the art and as further disclosed by the patent documents incorporated herein by reference. Yeast cell wall or an extract thereof may have a feed supplement comprising 0-15% moisture and 85-100% dry matter. The dry matter may comprise 10-65% protein, 0-25% fats, 0-3% phosphorus, 5-30% β-glucan, 5-35% mannans, and 0-15% ash. In an independent aspect, a commercial source of 3-1,3 (4) glucan and glucomannan derived from primary inactivated yeast (Saccharomyces cerevisiae) with the following chemical feed supplement can be used: moisture 2-5%; proteins 40-50%; fats 3-8%; phosphorus 0-2%; mannans 10-16%; β-1,3-(4) glucan 10-20%; and ash 2-12%.

In another independent aspect, the yeast cell wall or an extract thereof comprises moisture 1-7% and dry matter 93-99%, and the dry matter may comprise proteins 18-28%, fats 10-17%, phosphorus 0-2%, mannans 20-30%, β-1,3-(4) glucan 18-28%, and ash 2-5%.

In an independent aspect of the feed supplement, silica, glucan and mannans, and mineral clay are combined at 1-40%, 0.5-25% and 40-92% by weight, respectively. In an independent aspect of the feed supplement and/or combination, β-1,3 (4)-endoglucanohydrolase, diatomaceous earth, yeast cell wall or an extract thereof, and mineral clay are combined at 0.05-3%, 1-40%, 1-20% and 40-92% by weight, respectively. In an independent feed supplement and/or combination, β-1,3 (4)-endoglucanohydrolase, diatomaceous earth, yeast cell wall or an extract thereof, and mineral clay are combined at 0.1-3%, 5-40%, 2-15% and 40-80% by weight, respectively. In another independent aspect of the feed supplement and/or combination, β-1,3 (4)-endoglucanohydrolase, diatomaceous earth, yeast cell wall or an extract thereof, and mineral clay are combined at 0.1-3%, 30-40%, 4-15% and 50-65% by weight, respectively.

The feed supplement may further comprise one or more additional components. Additional components may be used for any desired purpose, such as a substantially biologically inert material added, for example, as a filler, or to provide a desired beneficial effect. For example, the feed supplement may include a carbonate (including a metal carbonate such as calcium carbonate); a trace mineral, such as, but not limited to, chloride, fluoride, iodide, chromium, copper, zinc, iron, magnesium, manganese, molybdenum, phosphorus, potassium, sodium, sulfur, selenium, or a combination thereof; a bulking agent; a micro tracer, such as iron particles coated with a dye; yeast; allicin; alliin; allinase; algae; a polyphenol or plant material comprising polyphenol; a carrier; a colorant; a taste enhancer; a preservative; an oil; a vitamin; a sorbic acid or a salt thereof; or a combination thereof. The yeast may be yeast culture, active yeast, a live yeast, a dead yeast, yeast extract, or a combination thereof. The preservative may be benzoic acid or a salt thereof, e.g. sodium benzoate; lactic acid or a salt thereof, e.g. sodium lactate, potassium lactate or calcium lactate; propionic acid or a salt thereof, e.g. sodium propionate; ascorbic acid or a salt thereof, e.g. sodium ascorbate; gallic acid or a salt thereof e.g. sodium gallate; sulfur dioxide and/or sulfites; nitrites; nitrates; choline, or a salt thereof, such as an anion salt of choline, e.g. choline halide, such as chloride, bromide, iodide, fluoride, or choline hydroxide; or any combination thereof. The oil may be mineral oil, corn oil, soybean oil, or a combination thereof. The sorbic acid or salt thereof may be potassium sorbate, sodium sorbate, ammonium sorbate, or a combination thereof. The vitamin may be vitamin A, vitamin B₁, vitamin B₂, vitamin B₃, vitamin B₅, vitamin B₆, vitamin B₁₂, vitamin C, vitamin D, vitamin E, vitamin K, or a combination thereof.

Additionally, or alternatively, the additional components may comprise corn, soybean meal, wheat, wheat fiber, barley, rye, rice hulls, canola, limestone, salt, distillers dried grains with solubles (DDGS), dicalcium phosphate, sodium sesquicarbonate, methionine source, lysine source, L-threonine, biotin, folic acid, kelp, menadione dimethylpyrimidinol bisulfite, calcium aluminosilicate, or any combination thereof.

Additional information concerning feed supplement and/or additional components can be found in PCT application No. PCT/US2015/053439, and U.S. application Ser. Nos. 15/359,342, 14/699,740, 14/606,862, and 62/449,959 each of which is incorporated herein by reference in its entirety.

In some aspects, the feed supplement does not comprise additional components. In other aspects, the feed supplement comprises from greater than zero to 40% or more by weight additional components, such as from 0.1% to 40% by weight, or from 0.2% to 35% by weight additional components. In certain aspects, the feed supplement comprises from 0.1% to 5% by weight additional components, such as from 0.2% to 3% by weight. In other aspects, the feed supplement comprises from 5% to 20% by weight additional components, such as from 10% to 15% by weight. And in further aspects, the feed supplement comprises from 20% to 40% by weight additional components, such as from 30% to 35% by weight additional components.

In some aspects, the feed supplement comprises, consists essentially of, or consists of, silica, mineral clay, glucan, mannans, and endoglucanohydrolase; silica, mineral clay, glucan, mannans, endoglucanohydrolase, micro tracers and mineral oil; silica, mineral clay, glucan, mannans, endoglucanohydrolase, micro tracers, mineral oil, and vitamins; silica, mineral clay, glucan, mannans, endoglucanohydrolase, micro tracers, mineral oil, vitamins, and potassium sorbate; silica, mineral clay, glucan, mannans, endoglucanohydrolase, vitamins, and active yeast; silica, mineral clay, glucan, mannans, endoglucanohydrolase, micro tracers, mineral oil, and active yeast; silica, mineral clay, glucan, mannans, endoglucanohydrolase, and mineral oil; silica, mineral clay, glucan, mannans, endoglucanohydrolase, vitamins, and calcium carbonate; silica, mineral clay, glucan, mannans, endoglucanohydrolase, micro tracers, and wheat fiber; or silica, mineral clay, glucan, mannans, endoglucanohydrolase, and micro tracers. In any of these aspects, the glucan and mannans may be provided by yeast, yeast cell wall, or yeast cell wall extract.

In some aspects, the feed supplement does not comprise a peroxide compound. In some aspects, the feed supplement does not comprise hydrogen peroxide. In some aspects, the feed supplement does not comprise carbamide peroxide. In some aspects, the feed supplement does not comprise urea. In some aspects, the feed supplement does not comprise hydrogen peroxide and urea.

In certain aspects, the feed supplement is a powdered supplement. In other aspects, the feed supplement is a granulated supplement. The granulated feed supplement may comprise silica, mineral clay, glucan and/or mannans, and optionally endoglucanohydrolase as discussed above. The granulated feed supplement may have a bulk loose density of from 40 lb/ft³ to 150 lb/ft³. In some aspects, each granule in the granular composition comprises silica, mineral clay, glucan and/or mannans, and optionally endoglucanohydrolase, in relative amounts substantially the same as a relative amount of each ingredient in the composition as whole. Each granule in the granular composition may comprise, consist essentially of, or consist of, silica, mineral clay, glucan, mannans and endoglucanohydrolase. Alternatively, or additionally, each granule may comprise a substantially homogenous blend of silica, mineral clay, glucan and mannans, and optionally endoglucanohydrolase. The composition may comprise greater than 40% by weight granules having at least one dimension between 0.149 mm (100 mesh, U.S. standard mesh size) and 4.76 mm (4 mesh), and in some aspects, the composition comprises greater than 90% by weight granules having at least one dimension between 0.149 mm (100 mesh) and 2 mm (10 mesh). And/or the composition may comprise from greater than 0% to 100% granules by weight and from 0% to no more than 60%, such as no more than 10%, particles by weight, the granules having at least one dimension between 10 mesh (2.00 mm) and 100 mesh (0.149 mm), and the particles having at least one dimension of less than (i.e., smaller than) 100 mesh (0.149 mm). In any aspects, the granular composition comprises plural granules, each granule comprising silica, mineral clay, glucan and mannans, the granules having a size that when administered to an animal increases expression of interleukin 10 receptor β (IL10RB) for a time period subsequent to administration, such as subsequent to the onset of administration, relative to an animal that does not receive the composition. In some aspects the time period may be from the start of administration to from 28 days to at least 42 days. And/or the composition may have a mineral coefficient of variation of from 0% to 10%, or a proximate coefficient of variation of from 0% to 20%, or both. Additional information concerning the granular feed supplement can be found in WO 2018/140450 which is incorporated herein by reference in its entirety.

In some aspects, the feed supplement is administered daily to an animal at time intervals believed or determined to be effective for achieving a beneficial result. The feed supplement may be administered in a single dose daily or in divided doses throughout the day. The amount may be from greater than zero to 500 grams per animal per day, such as from 0.5 grams to 250 grams, from 5 grams to 200 grams, or from 10 grams to 70 grams per animal per day. Alternatively, the feed supplement may be fed or administered in an amount of from greater than zero to 1000 mgs or more per kilogram of the animal's body weight per day, such as from greater than zero to 500 mgs per kilogram body weight. In other aspects, the feed supplement is fed or administered per weight of animal feed. The feed supplement may be fed or administered in an amount of from greater than zero to 150 kg per ton (2000 pounds) of feed, such as from 0.1 kg to 100 kg per ton of feed. Alternatively, the feed supplement may be fed or administered in an amount of from greater than zero to 20 grams per kilogram of feed, such as from greater than zero to 10 grams of feed.

### 3. Additional DFM(s)

The disclosed Bacilli combination can be administered to an animal in the absence of additional DFMs or in combination with one or more additional DFMs. The additional DFM(s) may be any DFM suitable for administration to the particular animal. In some aspects, the animal is a poultry, particularly a chicken or a turkey, and the additional DFM is a DFM that provides a benefit to the poultry. In other aspects, the animal is a ruminants or an aquatic species. The additional DFM may be, by way of example and without limitation, an additional Lactobacillus, Enterococcus, Bifidobacterium, Propionibacterium, Streptococcus, Pediococcus, yeast, or a combination thereof.

Exemplary additional DFMs include, but are not limited to, Lactobacillus acidophilis, Lactobacillus plantarum, Lactobacillus casei, Lactobacillus gallinarum, Lactobacillus lactis, Lactobacillus salivarius, Lactobacillus reuteri, Lactobacillus bulgaricus, Bifidobacterium pseudolongum, Bifidobacterium thermophilium, Bifidobacterium longum, Bifidobacterium lactis, Bifidobacterium animalis, Bifidobacterium bifidum, Bifidobacterium infantis, Streptococcus bovis, Streptococcus faecium, Enterococcus faecium, Enterococcus faecalis, Enterococcus diacetylactis, Saccharomyces cerevisiae, Saccharomyces boulardii, Aspergillus oryzae, Aspergillus niger, Selenomonas ruminantium, Megasphaera elsdenii, Propionibacterium freudenreichii, Propionibacterium shermanii, Propionibacterium acidipropionici, Propionibacterium fensenii, Prevotella bryantii, Pediococcus acidilactici, Pediococcus cerevisiae, or a combination thereof.

In some aspects, the additional DFM may comprise an additional Bacillus species, such as, but not limited to, Bacillus alcalophilus, Bacillus alvei, Bacillus aminovorans, Bacillus aneurinolyticus, Bacillus anthracis, Bacillus aquaemaris, Bacillus atrophaeus, Bacillus boroniphilus, Bacillus brevis, Bacillus caldolyticus, Bacillus centrosporus, Bacillus cereus, Bacillus circulans, Bacillus firmus, Bacillus flavothermus, Bacillus fusiformis, Bacillus galliciensis, Bacillus globigii, Bacillus infernus, Bacillus larvae, Bacillus laterosporus, Bacillus lentus, Bacillus megaterium, Bacillus mesentericus, Bacillus mucilaginosus, Bacillus mycoides, Bacillus pantothenticus, Bacillus polymyxa, Bacillus pseudoanthracis, Bacillus schlegelii, Bacillus sphaericus, Bacillus sporothermodurans, Bacillus stearothermophilus, Bacillus thermoglucosidasius, Bacillus thuringiensis, Bacillus vulgatis, or Bacillus weihenstephanensis.

### F. Liquids

In some examples, the Bacilli combination is mixed with a liquid, for example, water. The Bacilli combination can be in the form of spores or liquid culture when mixed with water. In further aspects, the Bacilli combination with water is further combined with acid, for example acetic acid, or sorbic acid, and optionally also with glycerol. In one aspect, the Bacilli combination is mixed with water and acetic acid. In one aspect, the Bacilli combination is mixed with water and sorbic acid. In one aspect, the Bacilli combination is mixed with water and acetic acid and glycerol. And in one aspect, the Bacilli combination is mixed with water and sorbic acid and glycerol.

In some aspects, the Bacilli combination with water is further combined with an alkaline compound.

Liquid Bacilli combination compositions can have any suitable pH range, for example, a pH in the range of from 2 to 8, such as from 3 to 8, from 4 to 8, from 5 to 8, or from 5 to 7.

### VI. Beneficial Results from Administering the Bacilli Combination

Administration of a disclosed Bacilli combination to poultry may reduce pathogenic Enterococcus cecorum in the poultry. The pathogenic Enterococcus cecorum reduction may be from greater than zero to 30% or more, such as a reduction of from 5% to 30%, 10% to 30%, or from 15% to 30%, compared to an amount of pathogenic Enterococcus cecorum present in poultry that are not administered the combination. The reduction may be identified at various times, such as at 21, 28, 30, 35, and/or 42 days old for certain disclosed working aspects.

Administration of a disclosed Bacilli combination to poultry may reduce splenic and hepatic infiltration of pathogenic Enterococcus cecorum in poultry. The pathogenic Enterococcus cecorum reduction in the spleen and/or liver may be from greater than zero to 30% or more, such as a reduction of from 5% to 30%, 10% to 30%, or from 15% to 30%, compared to an amount of pathogenic Enterococcus cecorum present in the liver or spleen that are not administered the combination. The reduction may be identified at various times, such as at 21, 28, 30, 35, and/or 42 days old for certain disclosed working aspects.

Administration of a disclosed Bacilli combination to poultry may reduce infiltration of pathogenic Enterococcus cecorum in the vertebrae of poultry. The pathogenic Enterococcus cecorum reduction in the vertebrae may be from greater than zero to 30% or more, such as a reduction of from 5% to 30%, 10% to 30%, or from 15% to 30%, compared to an amount of pathogenic Enterococcus cecorum present in the vertebrae of poultry that are not administered the combination. The reduction may be identified at various times, such as at 21, 28, 30, 35, and/or 42 days old for certain disclosed working aspects.

Administering a Bacilli combination to an animal, such as poultry or cattle, has provided a substantial beneficial result when compared to administering each of the respective Bacillus species individually, or in combinations comprising only two species. These beneficial results are determined by considering, for example, feed conversion rate, average body weight, average body weight gain, body weight coefficient of variation, breast meat yield, bird mortality, lesion scores, Salmonella/E. coli/Clostridium perfingens (CP) incidence, and/or oocysts in fecal matter at various times during chick rearing.

Administration of a disclosed Bacilli combination to poultry may reduce E. coli in the poultry. The amount of E. coli reduction may be from greater than zero to 25% or more, such as a reduction of from 5% to 25%, or 10% to 22%, compared to an amount of E. coli present in poultry that are not administered the combination. The reduction may be identified at various times, such as at 21, 28, 30, 35, and/or 42 days old for certain disclosed working aspects.

Administration of a disclosed Bacilli combination to poultry may reduce Aerobic Plate Count (APC) in the poultry. The APC reduction may be from greater than zero to 20% or more, such as a reduction of from 5% to 20%, or 10% to 18%, compared to an amount of APC present in poultry that are not administered the combination. The reduction may be identified at various times, such as at 21 and/or 42 days old for certain disclosed working aspects.

Administration of a disclosed Bacilli combination to poultry may reduce Salmonella in the poultry. The Salmonella reduction may be from greater than zero to 65% or more, such as a reduction of from 25% to 65%, 35% to 65%, or from 45 to 65%, compared to an amount of Salmonella present in poultry that are not administered the combination. The reduction may be identified at various times, such as at 21, 28, 30, 35, and/or 42 days old for certain disclosed working aspects.

Administration of a disclosed Bacilli combination to poultry may reduce Clostridium perfringens in the poultry. The Clostridium perfringens reduction may be from greater than zero to 30% or more, such as a reduction of from 5% to 30%, 10% to 30%, or from 15% to 30%, compared to an amount of Clostridium perfringens present in poultry that are not administered the combination. The reduction may be identified at various times, such as at 21, 28, 30, 35, and/or 42 days old for certain disclosed working aspects.

Administration of a disclosed Bacilli combination to poultry may reduce fecal oocysts in the poultry. The oocysts reduction may be from greater than zero to 90% or more, such as a reduction of from 50% to 90%, or 75% to 90%, compared to an amount of oocysts present in poultry that are not administered the combination. The reduction may be identified at various times, such as at 21, 28, 30, 35 and/or 42 days old for certain disclosed working aspects.

Administration of a disclosed Bacilli combination to poultry may result in an improved lesion score in the poultry. The lesion score may be improved (i.e., lowered) by from greater than zero to 75% or more, such as from 25% to 75%, or from 30% to 75%, compared to a lesion score of poultry that are not administered the combination. The improvement may be identified at various times, such as at 21 and/or 42 days old for certain disclosed working aspects.

Administration of a disclosed Bacilli combination to poultry may result in an improved feed conversion rate in the poultry. The feed conversion rate may be improved (i.e., lowered) by from greater than zero to 10% or more, such as from 2% to 8%, or from 4% to 8%, compared to a feed conversion rate of poultry that are not administered the combination. The improvement may be identified at various times, such as poultry at 14, 21, 28, 30, 35, and/or 42 days old.

Administration of a disclosed Bacilli combination to poultry may result in a reduced poultry mortality rate. The mortality rate may be reduced by from greater than zero to 95% or more, such as from 50% to 95%, from 75% to 95% or from 80% to 95%, compared to a mortality rate of poultry that are not administered the combination. The improvement may be identified at various times, such as when the poultry are 14, 21, 28, 30, 35, and/or 42 days old.

### VII. EXAMPLES

### Example 1

### In Vitro Inhibition of Enterococcus cecorum by Bacillus Strains—Liquid Culture Inoculation

### Overview

An in vitro inhibition assay determines whether, and to what extent, one culturable microorganism is able to inhibit, or prevent the growth of, a second and/or third microorganism. To conduct the assay, two or more different microorganisms are grown near each other on the same petri plate containing a culture medium capable of supporting each microorganisms' growth. After incubation of the plate, one can observe the extent of growth prevention by the first microorganism against the second and/or third microorganism by measuring the radius from the colony edge of the first microorganism to the colony edge of the microorganism being inhibited. This halo shaped area, or radius, is referred to as the “inhibition zone” and can be an area where no growth of either microorganism is present on the culture medium or where growth has been inhibited. Therefore, the larger the zone of inhibition, the more effectively the first microorganism can prevent the growth of the second microorganism. The inhibition zone can be caused by secondary metabolites that are secreted by the first microorganism and leech into the surrounding culture medium. An in vitro inhibition assay can be employed to determine whether, and to what extent, a beneficial microorganism is able to prevent or inhibit the growth of a pathogenic microorganism. In Examples 1 and 2 herein, in vitro inhibition assays show the ability of beneficial Bacillus species to inhibit the growth of various Enterococcus cecorum strains.

### A. Materials and Methods

From a frozen culture, each Bacillus and field isolate Enterococcus cecorum strain was streaked plates containing trypticase soy agar (TSA) or blood agar (BA), respectively. Inoculated plates were then incubated overnight at 36±2° C., aerobically for Bacillus, and in 5±2% CO₂ for E. cecorum. Grown cultures were then held for up to 2 weeks at 2-8° C. Brain heart infusion agar (BHIA) plates were then inoculated with 4 spots of Bacillus each resulting in two plates total with four strains per plate. Each Bacillus spot was approximately 5 mm. These plates were then incubated aerobically overnight at 36±2° C. Approximately 5 mL of BHI broth was inoculated with one colony of each Enterococcus cecorum strain to be tested. This E. cecorum culture was then incubated in 5±2% CO₂ at 36±2° C. for 16-18 hours for a theoretical final concentration of 1×10⁹ CFU/mL. After overnight incubation, a very thin layer of molten BHIA was sprayed onto the BHIA plates containing the grown Bacillus spots and allowed to dry. A 1:1,000 dilution of each grown Enterococcus cecorum culture in 0.1% peptone for theoretical concentrations of 1×10⁶ CFU/mL was prepared. Three tubes containing molten 9 mL BHIA were seeded with 1 mL of each Enterococcus cecorum dilution to obtain 1×10⁵ CFU/mL and gently vortexed. 3 mL of the seeded molten BHIA was removed. The remaining 7 mL of suspension was slowly poured over the 2 BHIA plates containing the Bacillus spots. Care was taken to avoid pouring directly on top of the Bacillus spots. For a control, the third seeded BHIA was poured over the surface of a non-seeded BHIA plate and allow to dry. Once the plate dried, a disk containing 20 μL of a 200 μg/mL solution of enrofloxacin and a disk containing 20 μL of sterile distilled water were placed on the surface of the solidified medium. The plates were then incubated in 5±2% CO₂ overnight at 36±2° C. The zone of inhibition of Enterococcus cecorum was measured radially from the Bacillus spots.

### B. Results

TABLE 1
The zones of inhibition (mm) of specific Bacillus strains each against E. cecorum
strains.

| | Zone of inhibition (mm) per E. cecorum strain | | | | | |
|---|---|---|---|---|---|---|
| Organism and Strain | 39958 | 39959 | 39960 | 39961 | 39962 | 39973 |
| Alternate ID | 4141356L- | 4299826L- | 4347052L- | 4347054L- | 4347058L- | 4298206L- |
| | 4 | 1 | 3 | 5 | 9 | 3 |
| Bacillus pumilus | 6.2 | 3.1 | 3.0 | 5.7 | 2.5 | 3.5 |
| OBT-13216 | | | | | | |
| Bacillus licheniformis | 0.0 | 0.0 | 0.0 | * | 0.0 | 0.0 |
| OBT-618 | | | | | | |
| Bacillus subtilis | 7.1 | 7.5 | 6.3 | 7.8 | 3.3 | 6.7 |
| PR-0104 | | | | | | |
| Bacillus subtilis | 5.1 | 2.6 | 2.1 | 2.8 | 1.9 | 3.2 |
| Commercial Product 1 | | | | | | |
| Bacillus amyloliquefaciens | 3.3 | 2.7 | 3.1 | 2.8 | 2.0 | 2.3 |
| Commercial Product 2 | | | | | | |

*Unable to determine zone of inhibition due to spreading culture growth.

### Example 2

### In Vitro Inhibition of Enterococcus cecorum by Bacillus Strains—Spray Test

### A. Materials and Methods

Frozen liquid stock of Bacillus and the Enterococcus cecorum strains were thawed to room temperature. A loop of inoculant from each Bacillus species was individually plated onto trypticase soy agar (TSA) plates and grown aerobically at 36° C. overnight. Concurrently, liquid stocks of E. cecorum field isolates were thawed to room temperature and were plated onto brain heart infusion agar (BHIA) plates under an atmosphere containing 5% CO₂ at 36° C. overnight. Strains of Bacillus or E. cecorum were concurrently inoculated on separate BHIA plates and grown overnight at 36° C. in an aerobic atmosphere (Bacillus) or a 5% CO₂ atmosphere (E. cecorum).

After overnight incubation, diluted E. cecorum cultures were added to a liquid atomizer with a spray nozzle. The liquid atomizer was decontaminated and cleaned with peroxyacetic acid, followed by a flush with sterile water prior to use. Lids were removed from BHIA plates with Bacillus species previously grown overnight. Using the atomizer, a thin layer of E. cecorum diluted in sterile water were sprayed over the visible Bacillus colonies. The lids were replaced and BHIA plates were placed in an incubator at 36° C. overnight at an atmosphere containing 5% CO₂. After incubation, plates were visually inspected for Bacillus colony size and inhibition zone size, with size reported in millimeters.

### B. Results

Tables 2-5. Zone of inhibition measurements (mm) of various Bacillus species and strains against various Enterococcus cecorum strains.

TABLE 2

| Bacillus Species | Enterococcus cecorum strain | | | | | |
|---|---|---|---|---|---|---|
| and Strain | 4141356L-4 | 4141357L-5 | 4141367L-3 | 4141374L-10 | 4216216L-3 | 4216217L-4 |
| B. licheniformis | 5.5 | 5.5 | 5.5 | 5.5 | 5.5 | 5.5 |
| OBT-618 | | | | | | |
| B. pumilis | 6.1 | 6.0 | 6.3 | 6.8 | 5.5 | 4.8 |
| OBT-13216 | | | | | | |
| B. subtilis | 4.8 | 5.1 | 5.8 | 5.8 | 4.8 | 6.1 |
| PR-0104 | | | | | | |
| B. licheniformis | 3.9 | 5.5 | 5.3 | 6.0 | 4.5 | 4.9 |
| Control strain 1 | | | | | | |
| (051018-F1) | | | | | | |
| B. subtilis | 6.2 | 6.0 | 6.8 | 6.4 | 5.0 | 5.6 |
| Control Strain 2 | | | | | | |
| (OBT-12224) | | | | | | |

TABLE 3

| Bacillus Species | | | | | | |
|---|---|---|---|---|---|---|
| and Strain | 4299826L-1 | 4298204L-1 | 4298206L-3 | 4298207L-4A | 4298207L-4B | 4329433L-1 |
| B. licheniformis | 5.5 | 5.5 | 5.5 | 5.5 | 5.5 | 5.5 |
| OBT-618 | | | | | | |
| B. pumilis | 5.6 | 5.8 | 6.1 | 6.0 | 5.1 | 5.8 |
| OBT-13216 | | | | | | |
| B. subtilis | 6.0 | 5.1 | 5.8 | 6.3 | 6.0 | 5.8 |
| PR-0104 | | | | | | |
| B. licheniformis | 5.5 | 5.8 | 6.2 | 5.6 | 5.4 | 5.0 |
| Control strain 1 | | | | | | |
| (051018-F1) | | | | | | |
| B. subtilis | 5.9 | 5.4 | 6.0 | 5.1 | 5.3 | 5.3 |
| Control Strain 2 | | | | | | |
| (OBT-12224) | | | | | | |

TABLE 4

| Bacillus Species | | | | | | |
|---|---|---|---|---|---|---|
| and Strain | 4347050L-1 | 4347051L-2 | 4347052L-3 | 4347053L-4 | 4347054L-5 | 4347055L-6 |
| B. licheniformis | 5.5 | 5.5 | 5.5 | 5.5 | 5.5 | 5.5 |
| OBT-618 | | | | | | |
| B. pumilis | 4.8 | 4.7 | 5.3 | 6.6 | 6.5 | 6.0 |
| OBT-13216 | | | | | | |
| B. subtilis | 5.4 | 5.0 | 4.1 | 4.9 | 5.2 | 5.6 |
| PR-0104 | | | | | | |
| B. licheniformis | 4.5 | 5.3 | 5.0 | 6.2 | 6.0 | 5.7 |
| Control strain 1 | | | | | | |
| (051018-F1) | | | | | | |
| B. subtilis | 5.1 | 5.5 | 5.9 | 6.2 | 5.9 | 5.8 |
| Control Strain 2 | | | | | | |
| (OBT-12224) | | | | | | |

TABLE 5

| Bacillus Species | | | | | | |
|---|---|---|---|---|---|---|
| and Strain | 4347056L-7 | 4347057L-8 | 4347058L-9 | 4355506L-3 | 4355509L-6 | 4355510L-7 |
| B. licheniformis | 5.5 | 5.5 | 5.5 | 5.5 | 5.5 | 5.5 |
| OBT-618 | | | | | | |
| B. pumilis | 4.9 | 6.0 | 5.1 | 5.2 | 6.2 | 6.0 |
| OBT-13216 | | | | | | |
| B. subtilis | 5.5 | 5.5 | 5.9 | 6.1 | 5.4 | 5.4 |
| PR-0104 | | | | | | |
| B. licheniformis | 4.7 | 4.2 | 5.5 | 5.4 | 5.0 | 4.9 |
| Control strain 1 | | | | | | |
| (051018-F1) | | | | | | |
| B. subtilis | 5.9 | 5.4 | 5.0 | 5.5 | 5.1 | 5.9 |
| Control Strain 2 | | | | | | |
| (OBT-12224) | | | | | | |

### Example 3

### In vivo Inhibition of E. cecorum by Bacilli Combination

### A. Purpose

In this example, a Bacilli combination DFM is evaluated for its ability to prevent E. cecorum from colonizing broiler chickens systemically.

### B. Materials and Methods

### Experimental Ration

Rations consist of non-medicated commercial-type broiler starter and grower diets. Rations are fed ad libitum from date of chick arrival as follows: Starter DOT 0 until DOT 22, grower DOT 22 to DOT 36, and finisher from DOT 36 to DOT 41 (study termination). All feed is labeled with treatment code, diet phase and date of manufacture. Diets are fed as mash throughout the study. Experimental treatment feeds are prepared from a basal feed formulation. Treatment feeds are mixed to assure uniform distribution of respective test articles.

### Animal Information

Day-of-hatch Ross×Ross strain chicks are obtained and sexed at the hatchery. All birds are vaccinated (by spray cabinet) with a commercially approved coccidia vaccine at the normal recommended dosage. Only healthy appearing chicks are used in this study.

### Housing

Upon arrival, chicks are raised in 5×5 feet (1.5 m×1.5 m) floor pens (stocking density of 1.0 feet² per bird) on new litter (at placement), in a solid-sided barn, with dirt floors, and under ambient humidity. Litter is not replaced or amended during the course of this study. Feed and water are available ad libitum throughout the trial. Each pen contains 1 (one) tube feeder and Plasson drinkers (25 bird to feeder/drinker ratio).

Thermostatically controlled gas heaters are the primary heat source for the barn, if needed. One (1) heat lamp per pen provides supplemental heat during brooding. Fans and evaporative cooling pads are used to cool birds. Birds are provided a lighting program as per the primary breeder recommendations.

### Experimental Design and Treatments

### Numbers of Treatment Groups and Subjects.

Between 2 houses, there are approximately 32 pens containing approximately 800 chicks total where each pen contains approximately 25 birds. There are 4 treatment groups and 8 replication blocks.

### Treatment Types.

Four treatments are included: (1) a control with no preventative treatment, (2) the disclosed Bacillus combination treatment at dietary inclusion rate of 500,000 CFU/g of finished feed, (3) the disclosed Bacillus combination treatment at dietary inclusion rate of 1000,000 CFU/g of finished feed, and (4) the disclosed Bacillus combination treatment at dietary inclusion rate of 1,500,000 CFU/g of finished feed. These four treatment groups each receive an Enterococcus cecorum challenge at 14 days. Data collection is conducted at the starter, grower, and finisher periods. The Bacillus combination consists of Bacillus pumilus(30%), Bacillus subtilis (50%), and Bacillus licheniformis (20%).

### Bird Allocation and Pen Randomization

Eight hundred birds (800) are assigned to four (4) treatment groups with eight (8) replicate pens per treatment and 25 birds per pen. Treatment groups are assigned to pens within a block using randomized complete block. There is complete randomization and assignment of treatment groups to pens uses Random Permutation Tables. The study begins when birds are placed (day-of-hatch; DOT 0), at which time birds are allocated to experimental pens by random allocation and pen weights recorded. Only healthy birds are selected. On DOT 0, group body weights are recorded by pen. No birds are replaced during the course of the study.

### Challenge Administration, Sample Collection, and Analysis

1. Challenge Method. On DOT 14, all birds in all treatments are orally gavaged with 0.1 ml of Enterococcus cecorum SA3 (1.0×10⁷ CFU/bird).
2. Samples for Sepsis. On 28 days of age, four (4) birds per pen are necropsied and spleens aseptically collected into individual Whirlpak bags and immediately placed on ice for culture of challenge strain of E. cecorum.
3. Samples for Spondylitis. On DOT 41, 100 birds sampled per treatment are necropsied and the Free Thoracic Vertebra (FTV) is aseptically cultured with Stuart swabs. Also, the spleens are aseptically removed and placed in individual Whirlpak bags and frozen.
4. Mortality. Any mortality is necropsied after DOT 28 and spleens removed and frozen and vertebrae section placed in 10% buffered formalin.

### Feed Changes

Birds receive treatment appropriate feed from DOT 0 to DOT 41. On DOT 22 remaining starter feed is removed, weighed, and replaced with grower feed to DOT 36 and finisher through DOT 41. On DOT 41 remaining finisher feed is removed and weighed back.

### Body and Feed Weight

All birds are weighed by pen on DOT 0, 22, 36, and 41. Feed added to each pen's feeder is weighed at the beginning of each formulation period on DOT 0, 22, 36, and 41 (starter and grower). Any additional bags of feed are weighed (and documented) for each pen (as required) during each formulation period. Feed is distributed as needed to feeders from pre-weighed bags (assigned to each pen) throughout each period. Feed remaining in feeders (and feed bags if applicable) is weighed and disposed of on DOT 22, 36, and 41. Empty pan feeder weights are recorded prior to study initiation. The trial terminates on DOT 41.

### Management

Disease Control: No concomitant drug therapy is used during the study.

Monitoring: All birds are monitored for general flock condition, temperature, lighting, water, feed, litter condition, and unanticipated house conditions/events.

Mortality: Pens are checked daily for mortality. Birds are only culled to relieve suffering. Date and removal weight (kg) is recorded on all birds culled (or found dead). A gross necropsy is performed on all dead or culled birds to determine the bird sex and probable cause of death.

### Data Management

Data management and statistical analysis for culture and lesions of weight gain, feed consumption, and feed conversion, and lesion score results is performed.

Performance. Performance means for pen weight gain, feed consumption, feed conversion (adjusted for mortality: feed consumed/final live weight+mortality weight) are calculated.

Spleen Cultures. Number of spleen cultures positive for E. cecorum are compared with significance testing performed using Fisher's exact test with a cut-off set at P=0.05.

Osteochondrosis (OCD) and Spinal Lesions. Mean OCD scores are compared among treatments using non-parametric Kruskal-Wallis test and lesion prevalences compared to control using Fisher's exact test. Significances will be set at P=0.05.

### Example 4

### Prevention of Enterococcus cecorum Induced Spondylitis (Kinkyback) Using Direct Fed Microbial

### Experimental Design

Enterococcus cecorum has been demonstrated to be the principal bacteria most frequently isolated from cases of spondylitis of the free thoracic vertebrae (FTV). Commonly referred to as kinkyback in broilers, E. cecorum can be a normal flora bacteria found in the intestines of broiler chickens. In this study, a direct fed microbial and an autogenous vaccine was evaluated for its ability to prevent E. cecorum from colonizing the broilers systemically.

### Materials and Methods

### Experimental Ration

Rations consisted of non-medicated commercial-type broiler starter and grower diets compounded according to NRC guidelines and contained feedstuffs commonly used in the United States. Rations were fed ad libitum from date of chick arrival as follows: Starter-DOT 0 until DOT 22, grower DOT 22 to DOT 32, and finisher from DOT 32 to DOT 42 (study termination). Diets were fed as crumbles (starter) or pellets (grower and finisher) throughout the study. Diet formulations were included with source data. Experimental treatment feeds were prepared from a basal feed formulation. Quantities of all basal feed and test articles included in treatment group batches were documented and included with source data files.

### Animal Information

Day-of-hatch chicks were obtained and sexed at the hatchery. Upon arrival all birds were vaccinated (by spray cabinet) with a commercially approved coccidia vaccine at the normal recommended dosage. Only healthy appearing chicks were used in this study.

### Housing

Upon arrival chicks were raised in 5×5 feet (1.5 m×1.5 m) floor pens (stocking density of 1.0 feet² per bird) on new litter (at placement), in a solid-sided barn, with dirt floors, and under ambient humidity. Litter was not replaced or amended during the course of this study. Feed and water were available ad libitum throughout the trial. Each pen contained 1 (one) tube feeder and Plasson drinkers (25 bird to feeder/drinker ratio).

Thermostatically controlled gas heaters were the primary heat source for the barn (if needed). One (1) heat lamp per pen provided supplemental heat during brooding. Fans and evaporative cooling pads were used to cool birds. Birds were provided a lighting program as per the primary breeder recommendations. The pen diagram was documented and included in final report with source data.

TABLE 6
Treatments

| Treatment | | Inclusion | | | Challenge |
|---|---|---|---|---|---|
| ID | Treatment Description | Starter | Grower | Finisher | (04 Day) |
| T1 | Challenge Control | — | — | — | Yes |
| T2 | Disclosed composition- | 0.25 | 0.25 | 0.25 | Yes |
| | 500,000 CFU/g of feed | lb/U.S. ton | lb/U.S. ton | lb/U.S. ton | |
| T3 | Disclosed composition- | 0.5 lb/U.S. | 0.5 lb/U.S. | 0.5 lb/U.S. | Yes |
| | 1,000,000 CFU/g of feed | ton | ton | ton | |
| T4 | Disclosed composition- | 0.75 | 0.75 | 0.75 | Yes |
| | 1,500,000 CFU/g of feed | lb/U.S. ton | lb/U.S. ton | lb/U.S. ton | |

The aspect of the disclosed composition used in this study included Bacillus pumilus (30%), Bacillus subtilis (50%), and Bacillus licheniformis (20%) as the only active (DFM) components.

### Bird Allocation and Pen Randomization

Birds were assigned to the treatment groups with eight (8) replicate pens per treatment and 25 birds per pen. Treatment groups were assigned to pens within a block using randomized complete block. SPRG completed randomization and assignment of treatment groups to pens using Random Permutation Tables. The study began when birds were placed (day-of-hatch; DOT 0), at which time birds were allocated to experimental pens by random allocation and pen weights recorded. Only healthy birds were selected. On DOT 0, group body weights were recorded by pen. No birds were replaced during the course of the study.

### Challenge Administration and Sample Collection and Analysis

Challenge Method. On DOT 04, all birds in all treatments were orally gavaged with 0.1 ml of Enterococcus cecorum SA3 (4.0×10⁷ cfu/bird).

Samples for Sepsis. On 22 days of age, four (4) birds per pen were randomly selected as first to hand and were necropsied and spleens aseptically collected into individual Whirlpak bags and immediately placed on ice for culture of challenge strain of E. cecorum.

Samples for Spondylitis. On DOT 42, 100 birds were selected first to hand with twelve (12) from six (6) pens and fourteen (14) from remaining two (2) pens to be sampled per treatment were necropsied and the Free Thoracic Vertebra (FTV) was aseptically cultured with Stuart swabs. Also, the spleens were aseptically removed and placed in individual Whirlpak bags and frozen.

Mortality. Any mortality was necropsied after DOT 22 and spleens removed and frozen and vertebrae section placed in 10% buffered formalin.

### Feed Changes

Birds received treatment appropriate feed from DOT 0 to DOT 42. On DOT 22 remaining starter feed was removed, weighed, and replaced with grower feed to DOT 32 which was weighed, removed and replaced with finisher through DOT 42. On DOT 42 remaining finisher feed was removed and weighed back. All non-consumed feed was weighed and disposed of in the SPRG disposal pit.

### Body and Feed Weight

All birds were weighed by pen on DOT 0, 22, 32, and 42. Feed added to each pen's feeder was weighed at the beginning of each formulation period on DOT 0, 22, 32, and 42 (starter and grower). Any additional bags of feed were weighed (and documented) for each pen (as required) during each formulation period. Feed was distributed as needed to feeders from pre-weighed bags (assigned to each pen) throughout each period. Feed remaining in feeders (and feed bags if applicable) were weighed and disposed of on DOT 22, 32, and 42. Empty pan feeder weights were recorded prior to study initiation. The trial was terminated on DOT 42.

### Management

Disease Control: No concomitant drug therapy was used during the study. Disposable plastic boots were worn by study personnel required to enter pens (e.g., collect birds for study procedures). The disposable plastic boots were removed as the person stepped out of pen to avoid tracking fecal material throughout the facility. Disposable plastic boots were properly disposed of after use.

Monitoring: All birds were monitored for general flock condition, temperature, lighting, water, feed, litter condition, and unanticipated house conditions/events. Findings were documented twice daily during the regular working hours (one observation recorded final study day). On Saturday, Sunday, and observed holidays, one (1) observation was recorded.

Mortality: Pens were checked daily for mortality. Birds were only culled to relieve suffering. Date and removal weight (kg) were recorded on all birds culled (or found dead). A gross necropsy was performed on all dead or culled birds to determine the bird sex and probable cause of death.

Euthanasia and Disposition: Birds requiring euthanasia were euthanized by designated personnel via facility procedures and disposed of according to facility procedures.

Bird and Feed Disposition: All birds were disposed of by appropriate methods.

Scales: Scale maintenance and standardization procedures were followed prior to use.

Source Data Control and Handling: Data was recorded in indelible ink. Entries were legible and source data sheet signed (or initialed) and dated by individual recording entry. All source data errors and/or changes were initialed, dated, and a brief explanation (or error code) written directly on form.

Performance. Performance means for pen weight gain, feed consumption, feed conversion (adjusted for mortality: feed consumed/final live weight+mortality weight) were calculated. Statistical evaluation of the data was performed using a Statistix for Windows Program. The procedures used general linear procedures using ANOVA with a comparison of means using least significant differences (t-test) (LSD(T) at a significance level of 0.05.

Spleen Cultures. Number of spleen cultures positive for E. cecorum were compared with significance testing performed using Fisher's exact test with a cut-off set at P=0.05.

Osteochondrosis (OCD) and Spinal Lesions. Mean OCD scores were compared among treatments using non-parametric Kruskal-Wallis test and lesion prevalences compared to control using Fisher's exact test. Significances were set at P=0.05.

### Results

Performance Results from d0-d22. Performance results from d0-d22 are summarized in Table 7, and pre-planned contrasts of the treatment effects are summarized in Table 8. There was no significant overall effect of treatment with respect to feed intake (P=0.19), adjusted FCR (P=0.51), non-adjusted FCR (P=0.52), or weight gain (P=0.30).

TABLE 7
Estimated marginal means (SE) for performance measures by treatment
from d0-d22.

| | | Feed | | Non- | Weight |
|---|---|---|---|---|---|
| | No. | Intake | Adjusted | adjusted | Gain |
| Treatment | Pens | (kg/pen) | FCR | FCR | (kg/bird) |
| T1. Challenge | 8 | 28.79ª | 1.442ª | 1.444ª | 0.816ª |
| Control | | (0.51) | (0.031) | (0.032) | (0.015) |
| T2. 0.5M CFU/g | 8 | 30.43ª | 1.429ª | 1.430ª | 0.860ª |
| | | (0.51) | (0.031) | (0.032) | (0.015) |
| T3. 1.0M CFU/g | 8 | 28.99ª) | 1.372ª | 1.373ª | 0.853ª |
| | | (0.51) | (0.031) | (0.032) | (0.015) |
| T4. 1.5M CFU/g | 8 | 29.70ª | 1.436ª | 1.441ª | 0.845ª |
| | | (0.51) | (0.031) | (0.032) | (0.015) |
| ^{†}P-value | | 0.19 | 0.51 | 0.52 | 0.30 |

^{†}F-test of the overall treatment effect from a two-way ANOVA with fixed effects for treatment and block. Within columns, means with a superscript in common do not differ with a level of significance of 5% over all comparisons when using Tukey's multiple comparison procedure.

TABLE 8
P-values for pre-planned contrasts of the treatment effect from d0-d22.

| | Feed | | Non- | Weight |
|---|---|---|---|---|
| | Intake | Adjusted | adjusted | Gain |
| Contrast | (kg/pen) | FCR | FCR | (kg/bird) |
| Orthogonal polynomials for the | | | | |
| PP dose effect (T1, T2, T3, T4) | | | | |
| Linear | 0.57 | 0.58 | 0.64 | 0.24 |
| Quadratic | 0.36 | 0.22 | 0.21 | 0.085 |
| Cubic | 0.029 | 0.23 | 0.37 | 0.47 |

Performance Results from d0-d32. Performance results from d0-d32 are summarized in Table 9, and pre-planned contrasts of the treatment effects are summarized in Table 10. There was no significant overall effect of treatment with respect to feed intake (P=0.093), adjusted FCR (P=0.45), or non-adjusted FCR (P=0.50), but there was a significant treatment effect for weight gain (P=0.037).

TABLE 9
Estimated marginal means (SE) for performance measures by treatment
from d0-d32.

| | | Feed | | Non- | Weight |
|---|---|---|---|---|---|
| | No. | Intake | Adjusted | adjusted | Gain |
| Treatment | Pens | (kg/pen) | FCR | FCR | (kg/bird) |
| T1. Challenge | 8 | 55.23ª | 1.461ª | 1.462ª | 1.671ª |
| Control | | (0.92) | (0.016) | (0.016) | (0.027) |
| T2. 0.5M CFU/g | 8 | 58.86ª | 1.460ª | 1.469ª | 1.784^{b} |
| | | (0.92) | (0.016) | (0.016) | (0.027) |
| T3. 1.0M CFU/g | 8 | 57.02ª | 1.427ª | 1.435ª | 1.743ª^{b} |
| | | (0.92) | (0.016) | (0.016) | (0.027) |
| T4. 1.5M CFU/g | 8 | 56.07ª | 1.466ª | 1.474ª | 1.691^{ab} |
| | | (0.92) | (0.016) | (0.016) | (0.027) |
| ^{†}P-value | | 0.093 | 0.45 | 0.50 | 0.037 |

^{†}F-test of the overall treatment effect from a two-way ANOVA with fixed effects for treatment and block. Within columns, means with a superscript in common do not differ with a level of significance of 5% over all comparisons when using Tukey's multiple comparison procedure.

TABLE 10
P-values for pre-planned contrasts of the treatment effect from d0-d32.

| | Feed | | Non- | Weight |
|---|---|---|---|---|
| | Intake | Adjusted | adjusted | Gain |
| Contrast | (kg/pen) | FCR | FCR | (kg/bird) |
| Orthogonal polynomials for the | | | | |
| PP dose effect (T1, T2, T3, T4) | | | | |
| Linear | 0.87 | 0.82 | 0.99 | 0.88 |
| Quadratic | 0.019 | 0.23 | 0.33 | 0.004 |
| Cubic | 0.13 | 0.16 | 0.13 | 0.23 |

Performance Results from d0-d42. Performance results from d0-d42 are summarized in Table 11, and pre-planned contrasts of the treatment effects are summarized in Table 12. There was no significant overall effect of treatment with respect to adjusted FCR (P=0.19) or non-adjusted FCR (P=0.32), but there was a significant effect of treatment with respect to both feed intake (P=0.019) and weight gain (P=0.044). Although there was a significant overall effect of treatment for weight gain, none of the individual pairwise comparisons between treatments were statistically significant when using Tukey's procedure to control the overall type I error probability at 5%.

TABLE 11
Estimated marginal means (SE) for performance measures by treatment
from d0-d42.

| | | Feed | | Non- | Weight |
|---|---|---|---|---|---|
| | No. | Intake | Adjusted | adjusted | Gain |
| Treatment | Pens | (kg/pen) | FCR | FCR | (kg/bird) |
| T1. Challenge | 8 | 88.84ª | 1.578ª | 1.591ª | 2.601ª |
| Control | | (1.62) | (0.024) | (0.016) | (0.047) |
| T2. 0.5M CFU/g | 8 | 96.45^{b} | 1.593ª | 1.599ª | 2.773ª |
| | | (1.62) | (0.014) | (0.016) | (0.047) |
| T3. 1.0M CFU/g | 8 | 92.24^{ab} | 1.552ª | 1.562ª | 2.698ª |
| | | (1.62) | (0.014) | (0.016) | (0.047) |
| T4. 1.5M CFU/g | 8 | 89.46ª | 1.591ª | 1.608ª | 2.587ª |
| | | (1.62) | (0.014) | (0.016) | (0.047) |
| ^{†}P-value | | 0.019 | 0.19 | 0.32 | 0.044 |

^{†}F-test of the overall treatment effect from a two-way ANOVA with fixed effects for treatment and block. Within columns, means with a superscript in common do not differ with a level of significance of 5% over all comparisons when using Tukey's multiple comparison procedure.

TABLE 12
P-values for pre-planned contrasts of the treatment effect
from d0-d42.

| | Feed | | Non- | Weight |
|---|---|---|---|---|
| | Intake | Adjusted | adjusted | Gain |
| Contrast | (kg/pen) | FCR | FCR | (kg/bird) |
| Orthogonal polynomials | | | | |
| for the dose effect | | | | |
| (T1, T2, T3, T4) | | | | |
| Linear | 0.75 | 0.97 | 0.84 | 0.59 |
| Quadratic | 0.003 | 0.38 | 0.23 | 0.006 |
| Cubic | 0.079 | 0.040 | 0.077 | 0.33 |

Total Mortality and FHN Mortality. Total mortality percentages and FHN mortality percentages are summarized in Table 13. There was no significant difference between the treatments with respect to total mortality percent (P=0.50) or FHN mortality percent (P=0.45).

TABLE 13
Summary of total mortality percent and FHN mortality percent.

| | | Total Mortality | FHN Mortality |
|---|---|---|---|
| | | Percent | Percent |
| Treatment | No. Pens | (SE) | (SE) |
| T1. Challenge | 8 | 3.5ª (1.4) | 1.0ª (0.6) |
| Control | | | |
| T2. PP 0.5M CFU/g | 8 | 2.0ª (1.1) | 1.5ª (0.7) |
| T3. PP 1.0M CFU/g | 8 | 2.0ª (1.1) | 0.5ª (0.4) |
| T4. PP 1.5M CFU/g | 8 | 3.5ª (1.4) | 1.5ª (0.7) |
| ^{†}P-value | | 0.50 | 0.45 |

^{†}Total mortality and FHN mortality were compared between treatments using generalized linear models with a binomial family and logit link.
Within columns, percentages with a superscript in common do not differ with a level of significance of 5% over all comparisons using the Bonferroni procedure.

Spleen E. cecorum Prevalences. Spleen E. cecorum prevalences are summarized in Table 14. In a factorial analysis, there was a significant interaction between the effects of treatment and day (P<0.001). There was no significant difference between the treatments on day 22 (P=0.41), but there was a significant difference between the treatments on day 42 (P<0.001). On day 42, the prevalences in T2 and T5 were both significantly lower than that in T4.

TABLE 14
Summary of spleen E. cecorum prevalence (%) by treatment and day.
Four birds were sampled from each of 8 pens per group on day 22, and
12-14 birds were sampled from each of 8 pens per group on day 42.

| | No. positive/No. sampled (%) | | | |
|---|---|---|---|---|
| Treatment | Day 22 | Day 42 | | Total |
| T1. Challenge | 2/32 (6)^{a} | | | |
| Control | | | | |
| T2. 0.5M CFU/g | 7/32 (22)^{a} | 10/100 | (10)ª | 17/132 (13) |
| T3. 1.0M CFU/g | 4/32 (13)^{a} | 19/100 | (19)^{ab} | 23/132 (17) |
| T4. 1.5M CFU/g | 5/32 (16)^{a} | 30/100 | (30)^{b} | 35/132 (27) |

Within columns, percentages with a superscript in common do not differ with a level of significance of 5% over all comparisons using the Bonferroni procedure.

Free Thoracic Vertebrae (FTV) E. cecorum Prevalences. FTV E. cecorum prevalences are summarized in Table 15. There was a significant difference between treatments on day 42 (P=0.002), with T2 having a significantly lower prevalence than either T1 or T4.

TABLE 15
Summary of free thoracic vertebrae (FTV) E. cecorum prevalence by
treatment. Samples were collected from 12-14 birds in each of 8 pens
per group on day 42.

| | No. | No. positive | | |
|---|---|---|---|---|
| Treatment | samples | (%) | | ^{†}P |
| T1. Challenge | 100 | 8 | (8)^{b} | 0.002 |
| Control | | | | |
| T2. 0.5M CFU/g | 100 | 1 | (1)^{a} | |
| T3. 1.0M CFU/g | 100 | 5 | (5)^{ab} | |
| T4. 1.5M CFU/g | 100 | 8 | (8)^{b} | |

^{†}P-value for the overall effect of treatment from a GEE logistic regression model. Percentages with a superscript in common do not differ with a level of significance of 5% over all comparisons using the Bonferroni procedure.

### CONCLUSIONS

The study evaluated the disclosed composition (at 500,000 CFU/g, 1,000,000 CFU/g, and 1,500,000 CFU/g of feed) in the starter diet during an Enterococcus Cecorum (EC) challenge. Each group was represented by eight replicate pens of 25 male ross broiler chickens. E. cecorum was orally gavaged (4.0×10⁷ CFU/bird) on day of test 4 (DOT 4). On DOT 22 spleens were collected from four birds in each pen and submitted for culture. On DOT 42, spleens and free thoracic vertebrae swabs were collected from 100 birds in each treatment. Birds and feed were weighed on DOT 22, 32, and 42 to evaluate performance metrics.

### Performance Results

Previous E. cecorum challenge models indicate that the bacteria has a negative influence on growth and performance. Given the DOT 4 challenge, the birds may have experienced an impact on performance in the starter phase. On DOT 22, body weight gain in the 500,000 CFU/g group (T2) was greater numerically (0.860^{A}) than the challenge control (0.816^{A}) (Table 7). The disclosed composition at 500,000 CFU/g also had greater feed intake than the challenge control. Feed intake and body weight gain in the other groups were intermediate between the lowest inclusion of the disclosed composition and the challenge control group (Table 7). As the birds gain more weight, there is greater stress to leg joints, this became evident by more lame birds in all challenged treatments. On DOT 32, the disclosed composition at 500,000 CFU/g had significantly greater body weight gain than the challenge control (Table 9). The disclosed composition at 1,000,000 CFU/g was statistically intermediate to these groups. Feed intake reflected the body weight data at 32 days (Table 9). At 42 days, the disclosed composition at 500,000 CFU/g maintained greater body weight gain (2.773^{A}) compared to the challenge control (2.601^{B}) (Table 11). The disclosed composition at 1,000,000 CFU/g (2.698^{A}) had intermediate body weight gain. The disclosed composition at the lowest inclusion maintained the significantly greatest feed intake for the duration of the study. At termination, the disclosed composition at 1,000,000 had the numerically lowest non-adjusted FCR (Table 11). Overall mortality in the study was relatively low at 3.5% in the challenge group. The disclosed composition at 500,000 and 1,000,000 CFU/g both had numerically lower mortality at 2.0% (Table 13).

E. cecorum Culture Results

The Day 22 spleen samples give an indication the challenge was progressing normally. There were no significant differences in spleen E. cecorum positive (Table 14). On day 42, the E. cecorum prevalence in the 500,000 CFU/g treatment group had the lowest spleen E. cecorum prevalence.

Prevention of E. cecorum becoming colonized in the free thoracic vertebra (FTV) can result in lowered incidence of kinkyback in broilers. On day 42, the 500,000 CFU/g treatment group had a significant reduction in E. cecorum culture positive vertebra from the challenge control (Table 15).

### Overall

The 500,000 CFU/g of the disclosed composition consistently improved the birds performance with the Enterococcus cecoum challenge. This 500,000 CFU/g inclusion rate more effectively prevented the E. cecorum from translocating to the spleens and the vertebrae.

### Example 5

### Prevention of Enterococcus cecorum Induced Spondylitis (Kinkyback) in Broilers from Breeders Vaccinated with an Autogenous Vaccine and the Broilers Fed a Direct Fed Microbial

### Experimental Design

Enterococcus cecorum has been demonstrated to be the principal bacteria most frequently isolated from cases of spondylitis of the free thoracic vertebrae (FTV). Commonly referred to as kinkyback in broilers, E. cecorum can be a normal flora bacteria found in the intestines of broiler chickens. It has demonstrated broilers develop microfractures in the FTV and this often becomes infected by E. cecorum. In this study, a direct fed microbial and maternal antibodies from an autogenous vaccine was evaluated for its ability to prevent E. cecorum from colonizing the broilers systemically.

### Materials and Methods

### Experimental Ration

Broiler rations consisted of non-medicated commercial-type broiler starter and grower diets compounded according to NRC guidelines and contained feedstuffs commonly used in the United States. Hen diets were compounded to meet Cobb breeder guidelines. Rations were fed ad libitum from date of chick arrival as follows: Starter-DOT 0 until DOT 28, grower DOT 28 to DOT 35, and finisher from DOT 35 to DOT 42 (study termination). All feed was labeled with treatment code, diet phase and date of manufacture. Diets were fed as mash to hens and pelleted type of diet for broiler study throughout the study. Experimental treatments were prepared from a basal feed formulation. Quantities of all basal feed and test articles included in treatment group batches were documented and included with source data files. Treatment feeds were mixed to assure uniform distribution of respective test articles.

### Animal Information

### Pullet Hen Study

Two hundred forty (240) Cobb pullets and thirty (30) Cobb roosters, ten (10) weeks old were selected for the study. Upon placement, one hundred twenty (120) pullets of treatment T3 and T4 pullets were vaccinated in left breast and tagged with colored ear tags on wings. The T1 and T2 (control) pullets and DFM only pullets were wing tagged with a different color and not vaccinated. The room was divided in half. The birds were housed on concrete floor with new pine shavings. Water was either bell or nipple depending on source farm. Feeders were trough feeders with space according to breeder guidelines. Then when pullets were 18 weeks of age, T3 and T4 were again vaccinated a second time in right breast. At 17 weeks of age, day length was increased fifteen minutes per week to stimulate egg production. After 18 week vaccination, hand gather nests were placed in each room. From 23 weeks (6 weeks post second vaccination) for the next 3 weeks, eggs were gathered per room and set by treatment in 2 Jamesway incubators by T1/T2 and T3/T4. After hatch, chicks were placed per Table 18.

TABLE 16
Treatment Groups

| | | Hen | | |
|---|---|---|---|---|
| | | Vaccination | Timing | |
| ID | Treatment | (Y/N) | (wk age) | ml* |
| T1 | Challenge control | N | NA | NA |
| T2 | DFM Alone | N | NA | NA |
| T3 | Hen vaccine alone | Y | 12, 18 | 0.5 |
| T4 | DFM + Hen vaccine | Y | 12, 18 | 0.5 |

*Vaccine was administered intramuscularly in breast using a 18 gauge × 0.25 inch needle

The DFM composition used in this study included Bacillus pumilus (30%), Bacillus subtilis (50%), and Bacillus licheniformis (20%) as the only active (DFM) components.

### Kinky-Back Study

Day-of-hatch chicks were obtained from the Hatchery. The strain was Cobb×Cobb. Birds were sexed at the hatchery. Upon arrival all birds were vaccinated (by spray cabinet) with a commercially approved coccidia vaccine at the normal recommended dosage. Only healthy appearing chicks were used in this study.

### Housing

Upon arrival chicks were raised in 5×5 feet (1.5 m×1.5 m) floor pens (stocking density of 1.0 feet² per bird) on new litter (at placement), in a solid-sided barn, with dirt floors, and under ambient humidity. Litter was not replaced or amended during the course of this study. Feed and water were available ad libitum throughout the trial. Each pen contained 1 (one) tube feeder and Plasson drinkers (25 bird to feeder/drinker ratio).

Thermostatically controlled gas heaters were the primary heat source for the barn (if needed). One (1) heat lamp per pen provided supplemental heat during brooding. Fans and evaporative cooling pads were used to cool birds. Birds were provided a lighting program as per the primary breeder recommendations.

TABLE 17
Treatments

| | | DFM Inclusion Level | | | | |
|---|---|---|---|---|---|---|
| | | | | | Feed | |
| | | | | | Inclusion | |
| | | | | | Rate (All | |
| Treatment | Treatment | | | | diet | |
| ID | Description | Starter | Grower | Finisher | stages) | Challenge (01 Day) |
| T1 | Challenge | — | — | — | NA | Yes |
| | Control | | | | | |
| T2 | DFM Alone | 500,000 | 500,000 | 500,000 | 0.25 | Yes |
| | | CFU/g | CFU/g | CFU/g | pound/U.S. | |
| | | feed | feed | feed | ton | |
| T3 | Hen Vaccine | — | — | — | NA | Yes |
| | Alone | | | | | |
| T4 | DFM + Hen | 500,000 | 500,000 | 500,000 | 0.25 | Yes |
| | Vaccine | CFU/g | CFU/g | CFU/g | pound/U.S. | |
| | | feed | feed | feed | ton | |

### Bird Allocation and Pen Randomization

Eight hundred straight run (both sexes) birds (800) were assigned to four (4) treatment groups with eight (8) replicate pens per treatment and 25 birds per pen. Treatment groups were assigned to pens within a block using randomized complete block. The study began when birds were placed (day-of-hatch; DOT 0), at which time birds were allocated to experimental pens by random allocation and pen weights recorded. Only healthy birds were selected. On DOT 0, group body weights were recorded by pen. No birds were replaced during the course of the study.

### Challenge Administration and Sample Collection and Analysis

1. Challenge Method. On DOT 01, all birds in all treatments were orally gavaged with 0.1 ml of Enterococcus cecorum SA3 (1.0×10⁷ cfu/bird).
2. Samples for Sepsis. On 13 days of age, four (4) birds per pen were randomly selected by first to hand and necropsied and spleens aseptically collected into individual Whirlpak bags and immediately placed on ice for culture of challenge strain of E. cecorum.
3. Samples for Spondylitis. On DOT 42, 100 birds were selected first to hand with twelve (12) from six (6) pens and fourteen (14) from remaining two (2) pens to be sampled per treatment were necropsied and the Free Thoracic Vertebra (FTV) were aseptically cultured with Stuart swabs. Also, the spleens were aseptically removed and placed in individual Whirlpak bags and frozen.
4. Mortality. Any mortality were necropsied after DOT 28 and if lame or abscesses present the spleens or infected joints or vertebrae cultured.

### Feed Changes

Birds received treatment appropriate feed from DOT 0 to DOT 42. On DOT 28 remaining starter feed was removed, weighed, and replaced with grower feed to DOT 35 and remaining grower was weighed, removed and finisher through DOT 42. On DOT 43 remaining finisher feed was removed and weighed back. All non-consumed feed was weighed and disposed of.

### Body and Feed Weight

All birds were weighed by pen on DOT 0, 28, 35, and 42. Feed added to each pen's feeder was weighed at the beginning of each formulation period on DOT 0, 28, 35, and 42 (starter and grower). Any additional bags of feed were weighed (and documented) for each pen (as required) during each formulation period. Feed was distributed as needed to feeders from pre-weighed bags (assigned to each pen) throughout each period. Feed remaining in feeders (and feed bags if applicable) were weighed and disposed of on DOT 28, 35, and 42. Empty pan feeder weights were recorded prior to study initiation. The trial was terminated on DOT 42.

### Management

Disease Control: No concomitant drug therapy was used during the study. Disposable plastic boots were worn by study personnel required to enter pens (e.g., collect birds for study procedures). The disposable plastic boots were removed as the person stepped out of pen to avoid tracking fecal material throughout the facility. Disposable plastic boots were properly disposed of after use.

Monitoring: All birds were monitored for general flock condition, temperature, lighting, water, feed, litter condition, and unanticipated house conditions/events. Findings were documented twice daily during the regular working hours (one observation recorded final study day). On Saturday, Sunday, and observed holidays, one (1) observation was recorded.

Mortality: Pens were checked daily for mortality. Birds were only culled to relieve suffering. Date and removal weight (kg) were recorded on all birds culled (or found dead). A gross necropsy was performed on all dead or culled birds to determine the bird sex and probable cause of death.

Euthanasia and Disposition: Birds requiring euthanasia were euthanized by designated personnel via facility procedures and disposed of according to facility procedures.

Bird and Feed Disposition: All birds were disposed of by appropriate methods. All mortalities and remaining feeds (including mixer flushes) were buried.

Scales: Scale maintenance and standardization procedures were followed prior to use.

Source Data Control and Handling: Data was recorded in indelible ink. Entries were legible and source data sheet signed (or initialed) and dated by individual recording entry. All source data errors and/or changes were initialed, dated, and a brief explanation (or error code) written directly on form.

Performance. Performance means for pen weight gain, feed consumption, feed conversion (adjusted for mortality: feed consumed/final live weight+mortality weight) were calculated. Statistical evaluation of the data was performed using a Statistix for Windows Program. The statistical model contained the mail effects of vaccine and DFM and their interaction. For response criteria with significant vaccine×DFM interaction, means were separated by the PDIFF option with a Tukey-Kramer adjustment at a significant level of 0.05 and trend at 0.10.

Spleen Cultures. Number of spleen cultures positive for E. cecorum were compared with significance testing performed using Fisher's exact test with a cut-off set at P=0.05.

Osteochondrosis (OCD) and Spinal Lesions. Mean OCD scores were compared among treatments using non-parametric Kruskal-Wallis test and lesion prevalences compared to control using Fisher's exact test. Significances were set at P=0.05.

### Statistical Methods

Prevalences of E. cecorum in spleen and FTV samples were compared between treatments using generalized estimating equations (GEE) logistic regression with an exchangeable working correlation structure to account for the correlation between responses of birds from the same pen. Pairwise comparisons between treatments were performed using the Bonferroni procedure to limit the overall type I error probability to 5%. All statistical testing assumed a two-sided alternative hypothesis, and P<0.05 was considered significant. Analyses were performed using commercially available statistical software (Stata version 18.0, StataCorp LLC, College Station, TX).

### Results

Spleen E. cecorum Prevalences. Spleen E. cecorum prevalences are summarized in Table 18. In a factorial analysis, there was no significant effect of treatment (P=0.39), but there was a significant effect of day (P<0.001), with the prevalence on day 42 being higher than that on day 13. There was no significant interaction between the effects of treatment and day (P=0.12).

TABLE 18
Summary of spleen E. cecorum prevalence (%) by treatment and day. Four birds were sampled
from each of 8 pens per group on day 13, and 12-14 birds were sampled from each of 8 pens
per group on day 42.

| | No. positive/No. sampled (%) | | | | | |
|---|---|---|---|---|---|---|
| Treatment | Day 13 | | Day 42 | | Total | |
| T1. Challenge Control | 8/32 | (25) | 55/100 | (55) | 63/132 | (48)^{a} |
| T2. DFM Alone | 3/32 | (9) | 51/100 | (51) | 54/132 | (41)^{a} |
| T3. Hen Vaccine Alone | 5/32 | (16) | 62/100 | (62) | 67/132 | (51)^{a} |
| T4. DFM + Hen Vaccine | 1/32 | (3) | 58/100 | (58) | 59/132 | (45)^{a} |
| Total | 17/128 | (13)^{a} | 226/400 | (57)^{b} | 243/528 | (46) |

Marginal percentages with a superscript in common do not differ with a level of significance of 5% over all comparisons using the Bonferroni procedure.

Free Thoracic Vertebrae (FTV) E. cecorum Prevalences. FTV E. cecorum prevalences are summarized in Table 19. There was no significant difference between treatments on day 42 (P=0.098).

TABLE 19
Summary of free thoracic vertebrae (FTV) E. cecorum prevalence by
treatment. Samples were collected from 12-14 birds in each of 8 pens
per group on day 42.

| Treatment | No. samples | No. positive (%) | ^{†}P |
|---|---|---|---|
| T1. Challenge Control | 100 | 22 (22)^{a} | 0.098 |
| T2. DFM Alone | 100 | 20 (20)ª | |
| T3. Hen Vaccine Alone | 100 | 37 (37)^{a} | |
| T4. DFM + Hen Vaccine | 100 | 23 (23)^{a} | |

^{†}P-value for the overall effect of treatment from a GEE logistic regression model. Percentages with a superscript in common do not differ with a level of significance of 5% over all comparisons using the Bonferroni procedure.

Spleen E. faecalis Prevalences. Spleen E. faecalis prevalences are summarized in Table 20. Because there were no E. faecalis positive samples in T2, it was not possible to compare treatments using logistic regression. Using Fisher's exact test, which does not account for the correlation between responses of birds in the same pen, there was no significant overall effect of treatment (P=0.15), but there was a significant effect of day (P=0.001), with the prevalence on day 13 being higher than that on day 42. If separate comparisons of the treatments were performed on each day, there was no significant effect of treatment on either day 13 (P=0.21) or day 42 (P=1.00).

TABLE 20
Summary of spleen E. faecalis prevalence (%) by treatment and day. Four birds were
sampled from each of 8 pens per group on day 13, and 12-14 birds were sampled from
each of 8 pens per group on day 42.

| | No. positive/No. sampled (%) | | | | | |
|---|---|---|---|---|---|---|
| Treatment | Day 13 | | Day 42 | | Total | |
| T1. Challenge Control | 4/32 | (13) | 1/100 | (1) | 5/132 | (3.8)^{a} |
| T2. DFM Alone | 0/32 | (0) | 0/100 | (0) | 0/132 | (0.0)^{a} |
| T3. Hen Vaccine Alone | 1/32 | (3) | 1/100 | (1) | 2/132 | (1.5)ª |
| T4. DFM + Hen Vaccine | 2/32 | (6) | 0/100 | (0) | 2/132 | (1.5)^{a} |
| Total | 7/128 | (5)^{b} | 2/400 | (0.5)ª | 9/528 | (1.7) |

Marginal percentages with a superscript in common do not differ with a level of significance of 5% over all comparisons using the Bonferroni procedure.

Free Thoracic Vertebrae (FTV) E. faecalis/E. faecium Prevalences. E. faecalis was identified in 1/100 (1%) FTV sample from T4, and E. faecium was also identified in 1/100 (1%) FTV sample from T4. Neither E. faecalis nor E. faecium was identified in any FTV samples from T1, T2, or T3.

TABLE 21
Day 0 to 28 Performance Results

| | Feed | | Non- | |
|---|---|---|---|---|
| | Intake | Adjusted | Adjusted | Weight |
| Treatment | (Kg/Pen) | FCR* | FCR | Gain (kg) |
| 1. Challenge Control | 41.613^{A} | 1.449^{A} | 1.465^{A} | 1.353^{A} |
| No Hen Vaccination | | | | |
| 2. DFM Alone No Hen | 43.119^{A} | 1.425^{A} | 1.437^{A} | 1.396^{A} |
| Vaccination | | | | |
| 3. Hen Vaccine Alone | 42.069^{A} | 1.423^{A} | 1.437^{A} | 1.362^{A} |
| 4. DFM + Hen Vaccine | 42.062^{A} | 1.416^{A} | 1.429^{A} | 1.364^{A} |

*FCR adjusted for mortality

TABLE 22
Day 0 to 35 Performance Results

| | Feed | | Non- | |
|---|---|---|---|---|
| | Intake | Adjusted | Adjusted | Weight |
| Treatment | (Kg/Pen) | FCR* | FCR | Gain (kg) |
| 1. Challenge Control | 59.838^{B} | 1.538^{A} | 1.584^{A} | 1.876^{B} |
| No Hen Vaccination | | | | |
| 2. DFM Alone No Hen | 63.263^{A} | 1.494^{B} | 1.532^{A} | 2.013^{A} |
| Vaccination | | | | |
| 3. Hen Vaccine Alone | 61.038^{AB} | 1.510^{AB} | 1.617^{A} | 1.977^{AB} |
| 4. DFM + Hen Vaccine | 60.613^{AB} | 1.496^{AB} | 1.579^{A} | 1.937^{AB} |

*FCR adjusted for mortality

TABLE 23
Day 0 to 42 Performance Results

| | Feed | | Non- | | Percent | Percent | Percent |
|---|---|---|---|---|---|---|---|
| | Intake | Adjusted | Adjusted | Weight | Total | KB ** | FHN ** |
| Treatment | (Kg/Pen) | FCR* | FCR | Gain (kg) | Mortality | Mortality | Mortality |
| 1. Challenge | 78.469^{B} | 1.629^{A} | 1.733^{A} | 2.407^{A} | 10.50^{A} | 1.00^{A} | 2.50^{A} |
| Control | | | | | | | |
| No Hen | | | | | | | |
| Vaccination | | | | | | | |
| 2. DFM Alone | 84.663^{A} | 1.594^{A} | 1.672^{A} | 2.591^{A} | 7.50^{A} | 0.00^{A} | 1.50^{A} |
| No Hen | | | | | | | |
| Vaccination | | | | | | | |
| 3. Hen Vaccine | 80.325^{AB} | 1.615^{A} | 1.739^{A} | 2.507^{A} | 12.00^{A} | 1.50^{A} | 5.00^{A} |
| Alone | | | | | | | |
| 4. DFM + | 80.300^{AB} | 1.594^{A} | 1.702^{A} | 2.508^{A} | 10.50^{A} | 0.50^{A} | 2.50^{A} |
| Hen Vaccine | | | | | | | |

### Performance Data Analysis

Means for pen weight gain, feed consumption, feed conversion (adjusted for mortality): feed consumed/final live weight+mortality weight) and cause of mortality was calculated. The mortality was assessed by gross lesions on necropsy. Statistical evaluation of the data was performed using a STATISTIX for Windows program (Analytical Software, Tallahassee, FL). The procedures used were general linear procedures using ANOVA with a comparison of means using least significant difference (t-test) (LSD) (T)) at a significant level of 0.05.

### Study Interpretation

The current study evaluated ability to resist Enterococcus cecorum infection when broilers were fed a direct fed microbial and/or if the broilers came from progeny in which their parents were vaccinated with an autogenous vaccine for the homologous E. cecorum. Treatments consisted of a challenge control, a DFM-only treatment, hen vaccine-only, and DFM plus hen vaccine. Each treatment was composed of 8 replicate pens of 25 straight run Cobb broilers. The parents of the vaccinated treatment were vaccinated intramuscularly with an autogenous E. cecorum vaccine at 12 and 18 weeks of age. The in-feed microbial product (the disclosed DFM composition) was included at 0.25 lbs/ton (500,000 CFU/g of finished feed) across all phases in treatments that received the DFM. All broilers were vaccinated with a commercial coccidiosis vaccine (lx dose) by coarse spray. After vaccination, the chicks were orally gavaged with 0.1 mL of E. cecorum. (dose). Spleens were collected from four birds in each replicate on day 13 to monitor the progression of E. cecorum septicemia. On day 42, one hundred spleen and free thoracic vertebrae swab samples were collected from each treatment. These samples were submitted to NCSU for prevalence analysis. Birds and feed were weighed on day 0, 28, 35, and 42 to monitor performance metrics.

### Prevalence Data

The EC positive spleen prevalence was overall 13% a (17/128 birds) on day 13 and increased significantly by day 42 to 57%^{b} (226/400 birds) (Table 18). There were no significant differences in Enterococcus prevalence in spleen samples between treatments. However, both treatments with the DFM had numerically lower prevalence values compared to their relative control group (the same vaccine status). Free thoracic vertebrae swabs in the challenge control group were 22%^{a} positive on day 41 (Table 19). There were no significant differences in FTV swab prevalence on day 42. Similar to the spleen data, the DFM groups both had numerically lower prevalence compared to their respective control groups. Specifically, the challenge control (22%^{a}) was numerically greater than the DFM-alone (20%^{a}) and the Hen Vaccine alone (37%^{a}) was numerically greater than the DFM plus Hen Vaccine (23%^{a}).

Enterococcus faecalis was also identified in some collected samples. There was significantly greater prevalence at 13 days (5%^{b}) compared to the 42-day samples (0.5%^{a}) (Table 20). There were no significant differences in E. faecalis spleen prevalence between treatment. The challenge control was numerically greatest with over half of the total positive spleen samples, 5 of the 9 E. faecalis positive samples. Two birds were positive for other Enterococcus species (E. faecalis and E. faecium) in the free thoracic vertebrae samples. Both birds were in the vaccinated plus DFM treatment.

### Performance Results

There were no significant differences in broiler performance from 0 to 28 days (Table 21). Adjusted feed conversion was lower in the DFM-alone (1.494^{B}) compared to the challenge control (1.538^{B}) at 35 days, Table 22. The DFM-alone group was also heavier at 35 days than the challenge control. By 42 days, there was no significant difference in feed conversion or body weight gain between treatments (Table 23). Groups receiving DFM had numerically lower feed conversion, greater body weight gain, and lower mortality compared to their respective control groups.

Overall, there were no significant differences in Enterococcus cecorum prevalence between treatments. However, in all evaluated tissues there was numerical prevalence reduction in the groups that received the DFM. Similarly, DFM treatments had lower feed conversion at each interval compared to the treatments without this product. Broilers from vaccinated breeders did not significantly change the ability of Enterococcus cecorum SA3 to translocate and colonize tissues. Also, the DFM only treatment had the lowest kinkyback and femoral head necrosis mortality throughout study (Table 23). Based on the consistent numerical reduction in prevalence and the improved broiler performance, the DFM appears to have an influence on Enterococcus infection.

### VIII. Example Aspects

The following numbered paragraphs illustrate exemplary aspects of the disclosed technology.

Paragraph 1. A Bacilli combination, consisting essentially of Bacillus licheniformis, Bacillus pumilus, and Bacillus subtilis.

Paragraph 2. The Bacilli combination of paragraph 1, consisting of Bacillus licheniformis, Bacillus pumilus, and Bacillus subtilis.

Paragraph 3. The Bacilli combination of paragraph 1 or 2, wherein:
the Bacillus licheniformis comprises strain OBT 618;
the Bacillus pumilus comprises strain OBT 13216;
the Bacillus subtilis comprises strain PR 104; or
a combination thereof.

Paragraph 4. The Bacilli combination of paragraphs 1 or 2, wherein:
the Bacillus licheniformis is strain OBT 618;
the Bacillus pumilus is strain OBT 13216;
the Bacillus subtilis is strain PR 104; or
a combination thereof.

Paragraph 5. The Bacilli combination of any of paragraphs 1-3, wherein relative amounts of Bacillus species in the Bacilli combination comprises from 1% to 99% Bacillus lichenformis, from 1% to 99% Bacillus pumilus, and from 1% to 99% Bacillus subtilis, in amounts relative to each other, such that the total amount of Bacillus species is 100%.

Paragraph 6. The Bacilli combination of any of paragraphs 1-3, wherein the combination comprises 10² to 10¹¹ CFU/g of B. lichenformis, 10² to 10¹¹ CFU/g of B. pumilus, and 10² to 10¹¹ CFU/g of B. subtilis.

Paragraph 7. The Bacilli combination of any of paragraphs 1-6, wherein the combination comprises about 50% Bacillus subtilis, about 20% Bacillus lichenformis, and about 30% Bacillus pumilus in amounts relative to each other, such that the total amount of Bacillus species is 100%.

Paragraph 8. The Bacilli combination of any of paragraphs 1-6, wherein the combination is a composition.

Paragraph 9. A composition, comprising:
a Bacilli combination according to any one of paragraphs 1-8; and
an additional component.

Paragraph 10. The composition of paragraph 9, wherein the additional component comprises a carrier, a vitamin, a copper salt, allicin, alliin, alliinase, algae, a polyphenol or plant material comprising polyphenol, a feed supplement, an additional DFM, a feed, or a combination thereof.

Paragraph 11. The composition of paragraph 10, wherein the copper salt is copper chloride, copper bromide, copper iodide, copper sulfate, copper sulfite, copper bisulfite, copper thiosulfate, copper phosphate, monobasic copper phosphate, dibasic copper phosphate, copper hypophosphite, copper dihydrogen pyrophosphate, copper tetraborate, copper borate, copper carbonate, copper bicarbonate, copper metasilicate, copper citrate, copper malate, copper methionate, copper succinate, copper lactate, copper formate, copper acetate, copper butyrate, copper propionate, copper benzoate, copper tartrate, copper ascorbate, copper gluconate, or a combination thereof.

Paragraph 12. The composition according to any one of paragraphs 9-11 wherein the additional component comprises a vitamin.

Paragraph 13. The composition of paragraph 12, wherein the vitamin is vitamin A, vitamin B₁, vitamin B₂, vitamin B₃, vitamin B₅, vitamin B₆, vitamin B₁₂, vitamin C, vitamin D, vitamin E, vitamin K, or a combination thereof.

Paragraph 14. The composition of any one of paragraphs 9-13, wherein the composition further comprises an additional direct-fed microbial.

Paragraph 15. The composition of any of paragraphs 9-14, wherein the additional component comprises one or more of inulin, yucca, quillaja, silica, mineral clay, glucan, mannans, endoglucanohydrolase, or a combination thereof.

Paragraph 16. The composition of paragraph 15, wherein the additional component comprises Yucca schidigera, Quillaja saponaria, or a combination thereof.

Paragraph 17. The composition of paragraph 15 or 16, wherein the additional component comprises silica, mineral clay, glucan, and mannans.

Paragraph 18. A composition, comprising the Bacilli combination of any of paragraphs 1-8, and water.

Paragraph 19. The composition of paragraph 18, further comprising an acid.

Paragraph 20. The composition of paragraph 19, wherein the acid is acetic acid.

Paragraph 21. A composition for administration to poultry, comprising:
a Bacilli combination according to any one of paragraphs 1-8 and
a poultry feed.

Paragraph 22. A composition for administration to poultry, comprising:
the composition of any one of paragraphs 1-20; and
a poultry feed.

Paragraph 23. The composition of paragraph 21 or 22, wherein the poultry feed comprises plant material, a carbonate, sulfate, lactate, oxide, propionate, stearate, phosphate, mineral, copper species, sugar, salt, animal protein product, forage product, grain product, plant protein product, processed grain product, roughage product, molasses product, or combinations thereof.

Paragraph 24. The composition of any one of paragraphs 21-23, wherein the poultry feed comprises beet pulp, ground corn, corn syrup solids, plant fiber, rice hulls, soluble plant fiber, wheat middlings, microcrystalline cellulose, calcium carbonate, potassium carbonate, potassium sulfate, sodium sulfate, calcium lactate, calcium oxide, calcium propionate, calcium stearate, dicalcium phosphate dehydrate, monocalcium phosphate, sodium tripolyphosphate, tetra sodium pyrophosphate, dolomite, silicon dioxide, silica, limestone, vermiculite, bentonite, montmorillonite, kaolin, glucose, sucrose, dextrose, fructose, maltodextrin, sodium chloride, carrageenan, cellulose, guar gum, polyols, sodium alumino silicate, urea, biotin, folic acid, sodium sesquicarbonate, methionine source, lysine source, L-threonine, or combinations thereof.

Paragraph 25. The composition of any one of paragraphs 21-24, wherein the poultry feed comprises copper sulfate.

Paragraph 26. The composition of any one of paragraphs 21-25, wherein the composition comprises from 10² to 10¹¹ CFU/g of B. licheniformis, from 10² to 10¹¹ CFU/g of B. pumilus, and from 10² to 10¹¹ CFU/g of B. subtilis.

Paragraph 27. The composition of any one of paragraphs 21-25, wherein the composition comprises from 10⁴ to 10⁶ CFU/g of B. lichemformis, from 10⁴ to 10⁶ CFU/g of B. pumilus, and from 10⁴ to 10⁶ CFU/g of B. subtilis.

Paragraph 28. The composition of any one of paragraphs 21-25, wherein the composition comprises about 2×10⁵ CFU of Bacillus lichemformis, about 3×10⁵ of Bacillus pumilus, and about 5×10⁵ CFU of Bacillus subtilis for a final total concentration of 1×10⁶ CFU per gram of feed.

Paragraph 29. A method, comprising administering a Bacilli combination according to any one of paragraphs 1-8 to a subject.

Paragraph 30. The method according to paragraph 29, wherein the subject is livestock.

Paragraph 31. The method according to paragraph 30, wherein the livestock is poultry.

Paragraph 32. The method according to paragraph 30, wherein the livestock is ruminants.

Paragraph 33. The method according to paragraph 29, wherein the subject is an aquatic species.

Paragraph 34. A method of reducing bird mortality, lesion scores, Enterococcus cecorum, Salmonella species, Escherichia coli, and/or Clostridium perfingens (CP) incidence, and/or oocysts in fecal matter, comprising administering to poultry an effective amount of a composition of any one of paragraphs 1-28.

Paragraph 35. A composition according to any one of paragraphs 1-28 for use in a method for reducing bird mortality, lesion scores, Enterococcus cecorum, Salmonella species, Escherichia coli, and/or Clostridium perfingens (CP) incidence, and/or oocysts in fecal matter.

Paragraph 36. A use of a composition according to any one of paragraphs 1-28, in the manufacture of a medicament for reducing bird mortality, lesion scores, Enterococcus cecorum, Salmonella species, Escherichia coli, and/or Clostridium perfingens (CP) incidence, and/or oocysts in fecal matter.

In view of the many possible aspects to which the principles of the disclosure may be applied, it should be recognized that the illustrated aspects are only preferred examples of the disclosure and should not be taken as limiting the scope of the disclosure. Rather, the scope of the disclosure is defined by the following claims. We therefore claim as the disclosure all that comes within the scope and spirit of these claims.

We claim:

1. A composition, comprising Bacillus licheniformis, Bacillus pumilus, and Bacillus subtilis and no additional direct fed microbial.

2. The composition of claim 1, wherein the composition comprises from 25% to 75% Bacillus subtilis, from 10% to 50% Bacillus licheniformis, and from 10% to 50% Bacillus pumilus in amounts relative to each other such that the total amount of Bacillus species is 100%.

3. The composition of claim 1, wherein:
the Bacillus licheniformis is strain OBT 618;
the Bacillus pumilus is strain OBT 13216;
the Bacillus subtilis is strain PR 10⁴; or
a combination thereof.

4. A composition, comprising:
a first composition according to claim 1; and
an additional component.

5. The composition of claim 4, wherein the additional component is selected from a carrier, a vitamin, a copper salt, allicin, alliin, alliinase, algae, a polyphenol or plant material comprising polyphenol, a feed supplement, an additional DFM that is not a Bacillus species, a feed, or a combination thereof.

6. The composition of claim 4, wherein the additional component is a carrier, animal feed or a combination thereof.

7. The composition of claim 6, wherein the composition comprises the animal feed and the composition comprises from 1 to 5×10⁵ CFU/g of feed of Bacillus licheniformis, from 1 to 5×10⁵ CFU/g of feed of Bacillus pumilus, and from 2 to 8×10⁵ CFU/g of feed of Bacillus subtilis for a final total concentration of approximately 10⁶ CFU of Bacilli species per gram of the animal feed.

8. The composition of claim 4, wherein the additional component is a carrier.

9. The composition of claim 8, wherein the carrier comprises calcium carbonate, mineral oil, vegetable oil, dextrose, maltodextrin, or a combination thereof.

10. The composition of claim 5, wherein the feed supplement comprises yucca, quillaja, silica, mineral clay, glucan, mannans, endoglucanohydrolase, or a combination thereof.

11. The composition of claim 5, wherein the carrier comprises water.

12. The composition of claim 11, further comprising an acid.

13. The composition of claim 5, wherein the animal feed is a poultry feed.

14. A method, comprising administering the composition of claim 1 to an animal.

15. The method of claim 14, wherein the animal is poultry.

16. The method of claim 14, wherein the animal is a ruminant or an aquatic species.

17. The method of claim 14, wherein the composition of claim 1 is administered in a mixture with an animal feed.

18. The method of claim 17, wherein the mixture comprises approximately 10⁶ CFU Bacillus species per gram of the animal feed.

19. The method of claim 17, wherein the mixture comprises from 1 to 5×10⁵ CFU of Bacillus licheniformis per gram of the animal feed, from 1 to 5×10⁵ CFU Bacillus pumilus per gram of the animal feed, and from 2 to 8×10⁵ CFU Bacillus subtilis per gram of the animal feed.

20. The method of claim 14, wherein the method is a method for improving feed conversion of the animal, improving the efficiency of the animal, and/or reducing Enterococcus cecorum infection in an organ of the animal.
